SEQUENCE LISTING (70 sequences)

SEQ ID NO: 1 - DNA, 1806 bp, Thlaspi arvense
  source 1..1806: mol_type = genomic DNA
atgatatcacaaagagaagagagagaagagaagagagtgatgggagataagaaattgatttcatcgtcttcttctattgcctcggtttacgatactcgtaataataacaatcatcatcacccaccgtcttcctccgacgagatttctcagtttctccggcatattttcgaccgttcttctcctctcccttcttactattctccggcgacgatgacgacggcggcaatcggagtgcacggcgacccacatgcagacaacccccggagcttcgtttctcatccgccgtctgactctgcgctcccgtcgaagcgccccgctgattactctgaggttttaataggctccgccgttggatcagcctccgccgttggatcaggctcagccccgtgttttggtttctccggaggtaataacattgcccaaggaaacagctcagggactcgagtttcgtcttcttccgttggagctagcgggaatgacaccgacgagtacgattgcgaaagcgaggtctctctctctctatgtgcatgttctaaaagttcccatctttgtctgtttcctgagaaaatgttatactgtgactttctctaacggatctgtactttcttttctcaccattcaagtgagcaaattaaatttgccttttttttctgtgtgtgtgtttttttagtgaagtttgtgaatgttaataatgcacacagagtgtttgttgatttgcttgaatgaaatcaggaaggagtagaagctgtggttgatgatgatcttccctcaaagtctggtccttctcgtagctcatcaaagcgatgcagagctgctgaagttcataatttgtctgaaaaggttttttatttgctccttgtttttgttttctctcccaaaatcacattcctttttactcagagattgatgtgatcttgttctgacagaggaggagaagtaggatcaacgaaaaaatgaaagctttacaaagtctcatcccaaattcaaacaaggtaaaaatacatacaaatgctgaatcattctctcatttgtctcttgttattgtgtctgattatataatgtccattgcaatgcgttgatgattggtgggaagacggataaggcttcaatgcttgatgaagctatagagtatctgaaacagcttcagcttcaagtccaggtcacaaaatatccattctcaaaaagatatgatacattcacttttcccgaatcaatcttatgaacagattactctgtgttttgcagatgttgacaatgaggaatggaataaacttgcatcctctgtgcttacctggaactacattacacccattgcaactctctcaggttcgagggatgcctcaagaagcaaccaatgatcatctgcttaatcacaccaaccaattcggttcgacctctaacgcacctgagatgatcaacaccgtgccttcctcatactcgttggaaccttccgtccgcagtcactttggacctttccctctccttacttcacacgcggtgcgtggtttcataacacattttcaatctataaaccctagattcttgaaagctagtgttcttactagaaatttattgttttttcgtaaaggagatgagtcgagaaggtggactaactcatcacaggttgagcattggtcattccaacacaaacttaaccggtaaagtcttcctgatttctgaattctcgtgaagaagtttttaagacattgacaatgttaaaaatgttgcgacgtttgggtatttgcaggggcacaagctgtgtttaatggacaagaacaacctgacataaaagatcgacttacttga

SEQ ID NO: 2 - protein, 375 aa, Thlaspi arvense
MISQREEREEKRVMGDKKLISSSSSIASVYDTRNNNNHHHPPSSSDEISQFLRHIFDRSSPLPSYYSPATMTTAAIGVHGDPHADNPRSFVSHPPSDSALPSKRPADYSEVLIGSAVGSASAVGSGSAPCFGFSGGNNIAQGNSSGTRVSSSSVGASGNDTDEYDCESEEGVEAVVDDDLPSKSGPSRSSSKRCRAAEVHNLSEKRRRSRINEKMKALQSLIPNSNKTDKASMLDEAIEYLKQLQLQVQMLTMRNGINLHPLCLPGTTLHPLQLSQVRGMPQEATNDHLLNHTNQFGSTSNAPEMINTVPSSYSLEPSVRSHFGPFPLLTSHAEMSREGGLTHHRLSIGHSNTNLTGAQAVFNGQEQPDIKDRLT

SEQ ID NO: 3 - DNA, 1806 bp, synthetic construct
  source 1..1806: mol_type = other DNA
atgatatcacaaagagaagagagagaagagaagagagtgatgggagataagaaattgatttcatcgtcttcttctattgcctcggtttacgatactcgtaataataacaatcatcatcacccaccgtcttcctccgacgagatttctcagtttctctggcatattttcgaccgttcttctcctctcccttcttactattctccggcgacgatgacgacggcggcaatcggagtgcacggcgacccacatgcagacaacccccggagcttcgtttctcatccgccgtctgactctgcgctcccgtcgaagcgccccgctgattactctgaggttttaataggctccgccgttggatcagcctccgccgttggatcaggctcagccccgtgttttggtttctccggaggtaataacattgcccaaggaaacagctcagggactcgagtttcgtcttcttccgttggagctagcgggaatgacaccgacgagtacgattgcgaaagcgaggtctctctctctctatgtgcatgttctaaaagttcccatctttgtctgtttcctgagaaaatgttatactgtgactttctctaacggatctgtactttcttttctcaccattcaagtgagcaaattaaatttgccttttttttctgtgtgtgtgtttttttagtgaagtttgtgaatgttaataatgcacacagagtgtttgttgatttgcttgaatgaaatcaggaaggagtagaagctgtggttgatgatgatcttccctcaaagtctggtccttctcgtagctcatcaaagcgatgcagagctgctgaagttcataatttgtctgaaaaggttttttatttgctccttgtttttgttttctctcccaaaatcacattcctttttactcagagattgatgtgatcttgttctgacagaggaggagaagtaggatcaacgaaaaaatgaaagctttacaaagtctcatcccaaattcaaacaaggtaaaaatacatacaaatgctgaatcattctctcatttgtctcttgttattgtgtctgattatataatgtccattgcaatgcgttgatgattggtgggaagacggataaggcttcaatgcttgatgaagctatagagtatctgaaacagcttcagcttcaagtccaggtcacaaaatatccattctcaaaaagatatgatacattcacttttcccgaatcaatcttatgaacagattactctgtgttttgcagatgttgacaatgaggaatggaataaacttgcatcctctgtgcttacctggaactacattacacccattgcaactctctcaggttcgagggatgcctcaagaagcaaccaatgatcatctgcttaatcacaccaaccaattcggttcgacctctaacgcacctgagatgatcaacaccgtgccttcctcatactcgttggaaccttccgtccgcagtcactttggacctttccctctccttacttcacacgcggtgcgtggtttcataacacattttcaatctataaaccctagattcttgaaagctagtgttcttactagaaatttattgttttttcgtaaaggagatgagtcgagaaggtggactaactcatcacaggttgagcattggtcattccaacacaaacttaaccggtaaagtcttcctgatttctgaattctcgtgaagaagtttttaagacattgacaatgttaaaaatgttgcgacgtttgggtatttgcaggggcacaagctgtgtttaatggacaagaacaacctgacataaaagatcgacttacttga

SEQ ID NO: 4 - protein, 375 aa, synthetic construct
MISQREEREEKRVMGDKKLISSSSSIASVYDTRNNNNHHHPPSSSDEISQFLWHIFDRSSPLPSYYSPATMTTAAIGVHGDPHADNPRSFVSHPPSDSALPSKRPADYSEVLIGSAVGSASAVGSGSAPCFGFSGGNNIAQGNSSGTRVSSSSVGASGNDTDEYDCESEEGVEAVVDDDLPSKSGPSRSSSKRCRAAEVHNLSEKRRRSRINEKMKALQSLIPNSNKTDKASMLDEAIEYLKQLQLQVQMLTMRNGINLHPLCLPGTTLHPLQLSQVRGMPQEATNDHLLNHTNQFGSTSNAPEMINTVPSSYSLEPSVRSHFGPFPLLTSHAEMSREGGLTHHRLSIGHSNTNLTGAQAVFNGQEQPDIKDRLT

SEQ ID NO: 5 - DNA, 973 bp, Thlaspi arvense
  source 1..973: mol_type = genomic DNA
atgggcaatcccgacgacggtgatcgtcttcctcctccatcttcttccgacgaactctcgagcgttctccggcagattctgtcccgtgccccgataactcaaccttcgtcgtcaccaccgaggagagtcgtttcctccgctgaaatgttcgaccggaccttccctttcgttcccggcggagcggtttcttccgccgcctataaagtcgctggcgaagacaaatgtgctttcgaaaacaaggtaagctaacatttttaagctgtcgagaaacttcactcgcttcgtttatgaattaagctaacatttctttgtaatggtaacaacactaaagagaaatggaggagctaaacatcgaaattcgttgaagagaaacaatgatgcacaattccacaacttgtctgaaaaggttctgtcttttaatcttctaaagattctcgatttgagaaagaaaagcaattgtgattttaatttatagaatctgaaattatttgcagaggaggaggagcaagatcaacgagaaaatgaaagctttgcagaaactgatacccaattccaacaaggtaaatgaaaaaagttggaatctttctacttctgaatacaatcgtgagaaacaccgttatgcttttgtttgtttgtttgtagactgataaagcctcaatgctcgatgaagctatagagtatatgaagcagcttcaacttcaagtgcaggtttttggctttactaagatcatatacaaccaaattataattttttgtaaaactcagcgcttatttgatcatacaatggataatgcagactttagcagtcatgaatggtttaggcctaaacccaatgcgattaccaccaacacagacaaggatcaatgaggccttacacatgcagactctgcttggcggttctcactcgcttgttcaccgtgaaccacccgaagcaagtcaagaaatgtgcttttccgctgcggctcgtctttaa

SEQ ID NO: 6 - protein, 207 aa, Thlaspi arvense
MGNPDDGDRLPPPSSSDELSSVLRQILSRAPITQPSSSPPRRVVSSAEMFDRTFPFVPGGAVSSAAYKVAGEDKCAFENKRNGGAKHRNSLKRNNDAQFHNLSEKRRRSKINEKMKALQKLIPNSNKTDKASMLDEAIEYMKQLQLQVQTLAVMNGLGLNPMRLPPTQTRINEALHMQTLLGGSHSLVHREPPEASQEMCFSAAARL

SEQ ID NO: 7 - DNA, 977 bp, synthetic construct
  source 1..977: mol_type = other DNA
atgggcaatcccgacgacggtgatcgtcttcctcctccatcttcttccgacgaactctcgagcgttctccggcagattctgtcccgtgccccgataactcaaccttcgtcgtcaccaccgaggagagtcgtttcctccgctgaaatgttcgaccggaccttccctttcgttcccggcggagcggtttcttccgccgcctataaagtcgctggcgaagacaaatgtgctttcgaaaacaaggtaagctaacatttttaagctgtcgagaaacttcactcgcttcgtttatgaattaagctaacatttctttgtaatggtaacaacactaaagagaaatggaggagctaaacatcgaaattcgttgaagagaaacaatgatgcacaattccacaacttgtctgaaaaggttctgtcttttaatcttctaaagattctcgatttgagaaagaaaagcaattgtgattttaatttatagaatctgaaattatttgcagaggaggaggagcaagatcaacgagaaaatgaaagctttgcagaaactgatacccaattccaacaaggtaaatgaaaaaagttggaatctttctacttctgaatacaatcgtgagaaacaccgttatgcttttgtttgtttgtttgtagactgataaagcctcaatgctcgatgaagctatagagtatatgaagcagcttcaacttcaagtgcaggtttttggctttactaagatcatatacaaccaaattataattttttgtaaaactcagcgcttatttgatcatacaatggataatgcagactttagcagtcatgaatggtttaggcctaaacccaatgcgtctcattaccaccaacacagacaaggatcaatgaggccttacacatgcagactctgcttggcggttctcactcgcttgttcaccgtgaaccacccgaagcaagtcaagaaatgtgcttttccgctgcggctcgtctttaa

SEQ ID NO: 8 - protein, 205 aa, synthetic construct
MGNPDDGDRLPPPSSSDELSSVLRQILSRAPITQPSSSPPRRVVSSAEMFDRTFPFVPGGAVSSAAYKVAGEDKCAFENKRNGGAKHRNSLKRNNDAQFHNLSEKRRRSKINEKMKALQKLIPNSNKTDKASMLDEAIEYMKQLQLQVQTLAVMNGLGLISLPPTQTRINEALHMQTLLGGSHSLVHREPPEASQEMCFSAAARL

SEQ ID NO: 9 - DNA, 519 bp, Thlaspi arvense
  source 1..519: mol_type = genomic DNA
atgaattggaacaaacctaatgatctcatcacacaagaatacccctttctccacgatcctcatctcatgatagatccacctcccgaaaccctaagtcatttccagcccccgccgacacttttctccggtcacggaggggaggaagaagaagaagaagataatgaagaggaagagatggatgcgatgaaggagatgcagtacacgatcgctgccatgcagcccgtggacatcgatccagccaccgttcctaaaccgaaccgccgtaacgtaagggtaagcgacgacactcagacggtggtgactcgtcggcgtcgagaaaagataagcgagaagatccgaatattgaagaggatggtgccaggcggtgcgaagatggacacagcctccatgctcgacgaagccatccgttataccaagttcttgaaacggcaggtgaagcttcttcagcctcactctcagcttggagctcctatgtctgacccctcttgcctttgttattaccacaactcccaaacctaa

SEQ ID NO: 10 - protein, 172 aa, Thlaspi arvense
MNWNKPNDLITQEYPFLHDPHLMIDPPPETLSHFQPPPTLFSGHGGEEEEEEDNEEEEMDAMKEMQYTIAAMQPVDIDPATVPKPNRRNVRVSDDTQTVVARRRREKISEKIRILKRMVPGGAKMDTASMLDEAIRYTKFLKRQVKLLQPHSQLGAPMSDPSCLCYYHNSQT

SEQ ID NO: 11 - DNA, 519 bp, synthetic construct
  source 1..519: mol_type = other DNA
atgaattggaacaaacctaatgatctcatcacacaagaatacccctttctccacgatcctcatctcatgatagatccacctcccgaaaccctaagtcatttccagcccccgccgacacttttctccggtcacggaggggaggaagaagaagaagaagataatgaagaggaagagatggatgcgatgaaggagatgcagtacacgatcgctgccatgcagcccgtggacatcgatccagccaccgtttctaaaccgaaccgccgtaacgtaagggtaagcgacgacactcagacggtggtggctcgtcggcgtcgagaaaagataagcgagaagatccgaatattgaagaggatggtgccaggcggtgcgaagatggacacagcctccatgctcgacgaagccatccgttataccaagttcttgaaacggcaggtgaagcttcttcagcctcactctcagcttggagctcctatgtctgacccctcttgcctttgttattaccacaactcccaaacctaa

SEQ ID NO: 12 - protein, 172 aa, synthetic construct
MNWNKPNDLITQEYPFLHDPHLMIDPPPETLSHFQPPPTLFSGHGGEEEEEEDNEEEEMDAMKEMQYTIAAMQPVDIDPATVSKPNRRNVRVSDDTQTVVARRRREKISEKIRILKRMVPGGAKMDTASMLDEAIRYTKFLKRQVKLLQPHSQLGAPMSDPSCLCYYHNSQT

SEQ ID NO: 13 - DNA, 519 bp, synthetic construct
  source 1..519: mol_type = other DNA
atgaattggaacaaacctaatgatctcatcacacaagaatacccctttctccacgatcctcatctcatgatagatccacctcccgaaaccctaagtcatttccagcccccgccgacacttttctccggtcacggaggggaggaagaagaagaagaagataatgaagaggaagagatggatgcgatgaaggagatgcagtacacgatcgctgccatgcagcccgtggacatcgatccagccaccgttcctaaaccgaaccaccgtaacgtaagggtaagcgacgacactcagacggtggtggctcgtcggcgtcgagaaaagataagcgagaagatccgaatattgaagaggatggtgccaggcggtgcgaagatggacacagcctccatgctcgacgaagccatccgttataccaagttcttgaaacggcaggtgaagcttcttcagcctcactctcagcttggagctcctatgtctgacccctcttgcctttgttattaccacaactcccaaacctaa

SEQ ID NO: 14 - protein, 172 aa, synthetic construct
MNWNKPNDLITQEYPFLHDPHLMIDPPPETLSHFQPPPTLFSGHGGEEEEEEDNEEEEMDAMKEMQYTIAAMQPVDIDPATVPKPNHRNVRVSDDTQTVVARRRREKISEKIRILKRMVPGGAKMDTASMLDEAIRYTKFLKRQVKLLQPHSQLGAPMSDPSCLCYYHNSQT

SEQ ID NO: 15 - DNA, 519 bp, synthetic construct; identical to SEQ ID NO: 9
  source 1..519: mol_type = other DNA

SEQ ID NO: 16 - protein, 172 aa, synthetic construct
MNWNKPNDLITQEYPFLHDPHLMIDPPPETLSHFQPPPTLFSGHGGEEEEEEDNEEEEMDAMKEMQYTIAAMQPVDIDPATVPKPNRRNVRVSDDTQTVVTRRRREKISEKIRILKRMVPGGAKMDTASMLDEAIRYTKFLKRQVKLLQPHSQLGAPMSDPSCLCYYHNSQT

SEQ ID NO: 17 - DNA, 747 bp, Thlaspi arvense
  source 1..747: mol_type = genomic DNA
atggaagagggtgggagtagtcacgacgcagagagtagcaagaagatagggagagggaagatagagataaagaggatagagaacacaacgaatcgtcaagtaactttctgcaaacgacgcaatggtcttctcaagaaagcttatgagctctctgtcttgtgtgatgccgaagttgccctcgttatcttctccactcgtggccgtctctatgagtatgccaacaacagtgtgaagggtacaattgaaaggtacaagaaagcttgttcagatgccgtcaatcccccctccgtcaccgaagctaatactcagtactatcagcaagaagcctctaagcttcggaggcagattcgagacattcagaactcaaacaggcatattgttggggaatcacttggttccttgaacttcaaggaactcaaaaacctcgaaggacgccttgaaaaaggaattagccgcgtccgatccaagaagaatgagttgttagtggcagagattgagtatatgcagaagagggaaatggatttgcaacacgataacatgtacctgcgagctaagatatccgaaggcgtgaggttgaatccggaacagcacggatcgagtgtgatacaaggaacagcgatttacgaatccggtgtgtcttctcatgatcagtcgcagcattataatcggaactatattccagtgaaccttcttgaaccaaatcagcaattctccggtcaagaccaacctcctcttcaacttgtttaa

SEQ ID NO: 18 - protein, 248 aa, Thlaspi arvense
MEEGGSSHDAESSKKIGRGKIEIKRIENTTNRQVTFCKRRNGLLKKAYELSVLCDAEVALVIFSTRGRLYEYANNSVKGTIERYKKACSDAVNPPSVTEANTQYYQQEASKLRRQIRDIQNSNRHIVGESLGSLNFKELKNLEGRLEKGISRVRSKKNELLVAEIEYMQKREMDLQHDNMYLRAKISEGVRLNPEQHGSSVIQGTAIYESGVSSHDQSQHYNRNYIPVNLLEPNQQFSGQDQPPLQLV

SEQ ID NO: 19 - DNA, 747 bp, synthetic construct
  source 1..747: mol_type = other DNA
atggaagagggtgggaatagtcacgacgcagagagtagcaagaagatagggagagggaagatagagataaagaggatagagaacacaacgaatcgtcaagtaactttctgcaaacgacgcaatggtcttctcaagaaagcttatgagctctctgtcttgtgtgatgccgaagttgccctcgttatcttctccactcgtggccgtctctatgagtatgccaacaacagtgtgaagggtacaattgaaaggtacaagaaagcttgttcagatgccgtcaatcccccctccgtcaccgaagctaatactcagtactatcagcaagaagcctctaagcttcggaggcagattcgagacattcagaactcaaacaggcatattgttggggaatcacttggttccttgaacttcaaggaactcaaaaacctcgaaggacgccttgaaaaaggaattagccgcgtccgatccaagaagaatgagttgttagtggcagagattgagtatatgcagaagagggaaatggatttgcaacacgataacatgtacctgcgagctaagatatccgaaggcgtgaggttgaatccggaacagcacggatcgagtgtgatacaaggaacagcgatttacgaatccggtgtgtcttctcatgatcagtcgcagcattataatcggaactatattccagtgaaccttcttgaaccaaatcagcaattctccggtcaagaccaacctcctcttcaacttgtttaa

SEQ ID NO: 20 - protein, 248 aa, synthetic construct
MEEGGNSHDAESSKKIGRGKIEIKRIENTTNRQVTFCKRRNGLLKKAYELSVLCDAEVALVIFSTRGRLYEYANNSVKGTIERYKKACSDAVNPPSVTEANTQYYQQEASKLRRQIRDIQNSNRHIVGESLGSLNFKELKNLEGRLEKGISRVRSKKNELLVAEIEYMQKREMDLQHDNMYLRAKISEGVRLNPEQHGSSVIQGTAIYESGVSSHDQSQHYNRNYIPVNLLEPNQQFSGQDQPPLQLV

SEQ ID NO: 21 - DNA, 741 bp, Thlaspi arvense
  source 1..741: mol_type = genomic DNA
atggagggtggtgcgagtaatgaagtagcagagagcagcaagaagatagggagagggaagatagagataaagaggatagagaatactacgaatcgtcaagtaactttctgcaaacgacgcaatggtttgctcaagaaagcttacgagctctccgtcttgtgtgatgcggaggttgctctcgtcatattctccactcgaggtcgtctctacgagtacgccaacaacagtgtaagaggaacgatcgaaaggtacaagaaagcttgctccgacgccgtgaatcctccttccgtcaccgaagctaatactcagtattatcagcaagagtcgtcgaagctacggagacagattcgagacattcagaatctgaacagacacattcttggtgagtctcttggttccttgaatctcaaggaactaaagaacctcgaaggtaggcttgagaaaggcatcagtcgcgtccgctccaagaagcacgagatgttagttgcagagatagagtacatgcaaaaaagggaaatcgagctgcaaaacgataacatgtatctccgatccaagattacggaaagggcaggagtacagcagcaggaatcgagtgtgatacatcaaggaacggtttacgagtcgggtgtatcgtcttctcatcagactgagcagtataaccggagttatattccggttaatctgctcgaaccaaatccgaattcctccgaccaagaccaaccacctctccaacttgtctaa

SEQ ID NO: 22 - protein, 246 aa, Thlaspi arvense
MEGGASNEVAESSKKIGRGKIEIKRIENTTNRQVTFCKRRNGLLKKAYELSVLCDAEVALVIFSTRGRLYEYANNSVRGTIERYKKACSDAVNPPSVTEANTQYYQQESSKLRRQIRDIQNLNRHILGESLGSLNLKELKNLEGRLEKGISRVRSKKHEMLVAEIEYMQKREIELQNDNMYLRSKITERAGVQQQESSVIHQGTVYESGVSSSHQTEQYNRSYIPVNLLEPNPNSSDQDQPPLQLV

SEQ ID NO: 23 - DNA, 741 bp, synthetic construct
  source 1..741: mol_type = other DNA
atggagggtggtgcgagtaatgaagtagcagagagcagcaagaagatagggagagggaagatagagataaagaggatagagaatactacgaatcgtcaagtaactttctgcaaacgacgcaatggtttgctcaagaaagcttacgagctctccgtcttgtgtgatgcggaggttactctcgtcatattctccactcgaggtcgtctctacgagtacgccaacaacagtgtaagaggaacgatcgaaaggtacaagaaagcttgctccgacgccgtgaatcctccttccgtcaccgaagctaatactcagtattatcagcaagagtcgtcgaagctacggagacagattcgagacattcagaatctgaacagacacattcttggtgagtctcttggttccttgaatctcaaggaactaaagaacctcgaaggtaggcttgagaaaggcatcagtcgcgtccgctccaagaagcacgagatgttagttgcagagatagagtacatgcaaaaaagggaaatcgagctgcaaaacgataacatgtatctccgatccaagattacggaaagggcaggagtacagcagcaggaatcgagtgtgatacatcaaggaacggtttacgagtcgggtgtatcgtcttctcatcagactgagcagtataaccggagttatattccggttaatctgctcgaaccaaatccgaattcctccgaccaagaccaaccacctctccaacttgtctaa

SEQ ID NO: 24 - protein, 246 aa, synthetic construct
MEGGASNEVAESSKKIGRGKIEIKRIENTTNRQVTFCKRRNGLLKKAYELSVLCDAEVTLVIFSTRGRLYEYANNSVRGTIERYKKACSDAVNPPSVTEANTQYYQQESSKLRRQIRDIQNLNRHILGESLGSLNLKELKNLEGRLEKGISRVRSKKHEMLVAEIEYMQKREIELQNDNMYLRSKITERAGVQQQESSVIHQGTVYESGVSSSHQTEQYNRSYIPVNLLEPNPNSSDQDQPPLQLV

SEQ ID NO: 25 - DNA, 1320 bp, Thlaspi arvense
  source 1..1320: mol_type = genomic DNA
atgttacgggaatcagacggtgagatgagcttagagacgacgaactcgccgattagcagcggaacagagagctgcagcagtttcagccggttatctttcgacgcgccgccgtcaaccaccgcgattatccccgaggaagagagccttctctctattaaaccgcaccgatcctccgatttcgcatactcggagatccgacggcggcggaaacaaggcctcaccttccgagattttcgactcatgcgtcgaatcggcgccggagacatcgggaccgtgtacttgtgccgtctcgccggagacgaagaagagagccggagctcgtatttcgcgatgaaagtggtggacaaagaagcgcttgcgatgaagaagaagatgcacagagcagagatggagaagacgattctgaagatgcttgaccatccgtttttgccgactctttacgccgagtttgacgcatcgcatttctcttgcatcgtcatggagtattgctccggcggagatttgcactccctccgtcacaaacagctcaaccgcagattctccctttcctccgccagattttacgcggctgaagttcttgtggcgctggaatatctacacatgctgggtatcatctacagagatctgaagcctgaaaatatcttagttagatcggacggtcacattatgctctctgactttgatctctccttatgctccgactcaatcgcagccgttgaatcctccacatcttcaccggagaatcaaccccgttcttcccggcgccgactcactcgactctctaggatcttccaccgagtcttgcggtccaaaaaggttcagacgctcgaaccgaaccgtctctttgttgccgaaccggtcaccgctcggtccggttcgtttgttggtacgcatgaatacgtggcaccagaagtcgcctcaggtgggtctcatggaaatgccgttgactggtgggccttcggagtattcctctacgagatgatctacggccggactccattcgccgcgccgacgaatgacgtcatccttcgtaacatcgtgaagagaccgttgagtttcccgaccgattcgccgtcgacgatgttcgagcttcacgcgcggggattgatctccgggttgctcaacaaggatccgaacaaacgactcgggtcacggcgaggcgcggcggaggttaaagtgcatccgtttttcaaaggtctaaactttgcgctcattcgtacattaactccgccggagattccctccgaggtcaggataccgaagaaatcgtcgacgttcggtggtagagctagtaaaccagcggcgttcgattacttttga

SEQ ID NO: 26 - protein, 439 aa, Thlaspi arvense
MLRESDGEMSLETTNSPISSGTESCSSFSRLSFDAPPSTTAIIPEEESLLSIKPHRSSDFAYSEIRRRRKQGLTFRDFRLMRRIGAGDIGTVYLCRLAGDEEESRSSYFAMKVVDKEALAMKKKMHRAEMEKTILKMLDHPFLPTLYAEFDASHFSCIVMEYCSGGDLHSLRHKQLNRRFSLSSARFYAAEVLVALEYLHMLGIIYRDLKPENILVRSDGHIMLSDFDLSLCSDSIAAVESSTSSPENQPRSSRRRLTRLSRIFHRVLRSKKVQTLEPNRLFVAEPVTARSGSFVGTHEYVAPEVASGGSHGNAVDWWAFGVFLYEMIYGRTPFAAPTNDVILRNIVKRPLSFPTDSPSTMFELHARGLISGLLNKDPNKRLGSRRGAAEVKVHPFFKGLNFALIRTLTPPEIPSEVRIPKKSSTFGGRASKPAAFDYF

SEQ ID NO: 27 - DNA, 1320 bp, synthetic construct
  source 1..1320: mol_type = other DNA
atgttacgggaatcagacggtgagatgagcttagagacgacgaactcgccgattagcagcggaacagagagctgcagcagtttcagccggttatctttcgacgcgccgccgtcaaccaccgcgattatccccgaggaagagagccttctctctattaaaccgcaccgatcctccgatttcgcatactcggagatccgacggcggcggaaacaaggcctcaccttccgagattttcgactcatgcgtcgaatcggcgccggagacatcgggaccgtgtacttgtgccgtctcgccggagacgaagaagagagccggagctcgtatttcgcgatgaaagtggtggacaaagaagcgcttgcgatgaagaagaagatgcacagagcagagatggagaagacgattctgaagatgcttgaccatccgtttttgccgactctttacgccgagtttgacgcatcgcatttctcttacatcgtcatggagtattgctccggcggagatttgcactccctccgtcacaaacagctcaaccgcagattctccctttcctccgccagattttacgcggctgaagttcttgtggcgctggaatatctacacatgctgggtatcatctacagagatctgaagcctgaaaatatcttagttagatcggacggtcacattatgctctctgactttgatctctccttatgctccgactcaatcgcagccgttgaatcctccacatcttcaccggagaatcaaccccgttcttcccggcgccgactcactcgactctctaggatcttccaccgagtcttgcggtccaaaaaggttcagacgctcgaaccgaaccgtctctttgttgccgaaccggtcaccgctcggtccggttcgtttgttggtacgcatgaatacgtggcaccagaagtcgcctcaggtgggtctcatggaaatgccgttgactggtgggccttcggagtattcctctacgagatgatctacggccggactccattcgccgcgccgacgaatgacgtcatccttcgtaacatcgtgaagagaccgttgagtttcccgaccgattcgccgtcgacgatgttcgagcttcacgcgcggggattgatctccgggttgctcaacaaggatccgaacaaacgactcgggtcacggcgaggcgcggcggaggttaaagtgcatccgtttttcaaaggtctaaactttgcgctcattcgtacattaactccgccggagattccctccgaggtcaggataccgaagaaatcgtcgacgttcggtggtagagctagtaaaccagcggcgttcgattacttttga

SEQ ID NO: 28 - protein, 439 aa, synthetic construct
MLRESDGEMSLETTNSPISSGTESCSSFSRLSFDAPPSTTAIIPEEESLLSIKPHRSSDFAYSEIRRRRKQGLTFRDFRLMRRIGAGDIGTVYLCRLAGDEEESRSSYFAMKVVDKEALAMKKKMHRAEMEKTILKMLDHPFLPTLYAEFDASHFSYIVMEYCSGGDLHSLRHKQLNRRFSLSSARFYAAEVLVALEYLHMLGIIYRDLKPENILVRSDGHIMLSDFDLSLCSDSIAAVESSTSSPENQPRSSRRRLTRLSRIFHRVLRSKKVQTLEPNRLFVAEPVTARSGSFVGTHEYVAPEVASGGSHGNAVDWWAFGVFLYEMIYGRTPFAAPTNDVILRNIVKRPLSFPTDSPSTMFELHARGLISGLLNKDPNKRLGSRRGAAEVKVHPFFKGLNFALIRTLTPPEIPSEVRIPKKSSTFGGRASKPAAFDYF

SEQ ID NO: 29 - DNA, 1311 bp, Thlaspi arvense
  source 1..1311: mol_type = genomic DNA
atggctcgtcgtttcggacttcttgctatcttcttatgtgttcttttgatgctctcgtggtgcgaagctttgagtagcaacgttgatgatggatatggtcatgaagatggaagcttcgaatccgatagcttactcaaacttaagaacgacgacgacgacgttcttaccttgaaaagctccgataaaaccacttccgaatcatcaactgttagtgttaccgatttcggtgctaaaggagatggggaaaacgatgatactcaggccttcaagaaagcatggaagaaagcatgttcaacaaagggagttactagtttcttaattcctaaaggaaagacttatctccttaagtctactcgattcagaggcccatgcaaatctttacgtaactttcagatcctaggcactttatcagcatctacaaaacgatctgattataagaatgacagaaaccattggcttgtcttggaggacgttaacaatctatcactggatggcggctcgacgggaattattgatggcaacggaaaaatctggtggcaaaattcatgcaaaatcgaccaatctaagccatgcacaaaagccccaacggctcttactttctacaacttaaagaatttgaatgtgaagaatctgagagtgagaaatgcgcagcagattcagatttcgattgagaaatgcaacaatgttaacgtcaacaatgtcgagatcactgctcctgacgatagtcccaacaccgatggtattcacatcactaatacacaaaacattcgaatctccaattcagacattggcacaggtgatgattgcatatccattgaggatggatcccaaaatgttcaaatcaatgatttaacttgcggccccggtcacgggatcagcattgggagtttgggggatgacaattcgaaagcttatgtctcggggattaatgtagatggtgctaagctctcttctactgataatggagttagaattaaaacttaccagggaggatcaggaactgccaagaacattaaatttcaaaatattcgtatggaaaatgtcaagaatccaatcataatcgaccagaactactgcgacaaggacaaatgcgaagaacaagaatccgcggtgcaagtaaacaatgtggtgtaccggaacataaccggtacgagcgcaacggatgtggcgataatgtttaattgcagtgagaaatatccatgccaagggattgtgcttgagaacgtgaatatcgaaggaggaacagcttcttgcaaaaatgccaatgttaaggatcaaggcactgtatctcctcagtgctcttccacttga

SEQ ID NO: 30 - protein, 436 aa, Thlaspi arvense
MARRFGLLAIFLCVLLMLSWCEALSSNVDDGYGHEDGSFESDSLLKLKNDDDDVLTLKSSDKTTSESSTVSVTDFGAKGDGENDDTQAFKKAWKKACSTKGVTSFLIPKGKTYLLKSTRFRGPCKSLRNFQILGTLSASTKRSDYKNDRNHWLVLEDVNNLSLDGGSTGIIDGNGKIWWQNSCKIDQSKPCTKAPTALTFYNLKNLNVKNLRVRNAQQIQISIEKCNNVNVNNVEITAPDDSPNTDGIHITNTQNIRISNSDIGTGDDCISIEDGSQNVQINDLTCGPGHGISIGSLGDDNSKAYVSGINVDGAKLSSTDNGVRIKTYQGGSGTAKNIKFQNIRMENVKNPIIIDQNYCDKDKCEEQESAVQVNNVVYRNITGTSATDVAIMFNCSEKYPCQGIVLENVNIEGGTASCKNANVKDQGTVSPQCSST

SEQ ID NO: 31 - DNA, 1311 bp, synthetic construct; identical to SEQ ID NO: 29
  source 1..1311: mol_type = other DNA

SEQ ID NO: 32 - protein, 56 aa, synthetic construct
MARRFGLLAIFLCVLLMLSWCEALSSNVDDGYGHEMEASNPIAYSNLRTTTTTFLP

SEQ ID NO: 33 - DNA, 1311 bp, synthetic construct; identical to SEQ ID NO: 29
  source 1..1311: mol_type = other DNA

SEQ ID NO: 34 - protein, 55 aa, synthetic construct
MARRFGLLAIFLCVLLMLSWCEALSSNVDDGYGQMEASNPIAYSNLRTTTTTFLP

SEQ ID NO: 35 - protein, 373 aa, Arabidopsis thaliana
MISQREEREEKKQRVMGDKKLISSSSSSSVYDTRINHHLHHPPSSSDEISQFLRHIFDRSSPLPSYYSPATTTTTASLIGVHGSGDPHADNSRSLVSHHPPSDSVLMSKRVGDFSEVLIGGGSGSAAACFGFSGGGNNNNVQGNSSGTRVSSSSVGASGNETDEYDCESEEGGEAVVDEAPSSKSGPSSRSSSKRCRAAEVHNLSEKRRRSRINEKMKALQSLIPNSNKTDKASMLDEAIEYLKQLQLQVQMLTMRNGINLHPLCLPGTTLHPLQLSQIRPPEATNDPLLNHTNQFASTSNAPEMINTVASSYALEPSIRSHFGPFPLLTSPVEMSREGGLTHPRLNIGHSNANITGEQALFDGQPDLKDRIT

SEQ ID NO: 36 - protein, 297 aa, Glycine max
MAGDIGRALPPPDSEEFSTLFNQLLHNSPPLGMDPNHSPSDFTPHNTTININSNNNNNTVPSSPSNFNFSDPHHYIPASDATTFKQHNINHNNNHTPDFTSSHVEKSVEASKPVPPPRSSSKRSRAAEFHNLSEKRRRSRINEKMKALQNLIPNSNKTDKASMLDEAIEYLKQLQLQVQMLMMRNGLSLHPMSLPGGLRPMIMPQTGLNLDGSNGFQNSTCAIASSSNDESLVRHAFSFPKQCSISNKSIGVPSVKNIATSDTSSTFHPSIKDALYGNMPQPFMDTTKIGKPSPDVS

SEQ ID NO: 37 - protein, 188 aa, Oryza sativa
EAGGSSEPEAAAGARPRGGSGSKRSRAAEVHNLSEKRRRSKINEKMKALQSLIPNSNKTDKASMLDEAIEYLKQLQLQVQMLSMRNGVYLNPSYLSGALEPAQASQMFAALGGNNVTVVHPGTVMPPVNQSSGAHHLFDPLNSPPQNQPQSLILPSVPSTAIPEPPFHLESSQSHLRQFQLPGSSEVI

SEQ ID NO: 38 - protein, 314 aa, Solanum lycopersicum
MADPYRTNPHASSSLESEDMSSFFLNFLQGTPASSSATAAAGFYNRSGPAPVAESSSSLNFSDPGRFYAAEFKEGVENVFASAGLGECDGMNSANRREFLEDDKVDNFGFSSEECDGLDMPSDPTHPRSSKRSRSAEVHNLSEKRRRSRINEKLKALQNLIPNSNKTDKASMLDEAIEYLKQLQLQVQILTLRNGLSLYPGYVPGSLQSVQLPSGNEFDGRSFMLSANGGATLPVNREMPQTAFEISNQNPSGKPTITSHNTENAVALETTIQNHYGLLNHLASSKDMCRDNTLSRLHLDMSCSGNNSSSGVSS

SEQ ID NO: 39 - protein, 198 aa, Arabidopsis thaliana
MENGMYKKKGVCDSCVSSKSRSNHSPKRSMMEPQPHHLLMDWNKANDLLTQEHAAFLNDPHHLMLDPPPETLIHLDEDEEYDEDMDAMKEMQYMIAVMQPVDIDPATVPKPNRRNVRISDDPQTVVARRRRERISEKIRILKRIVPGGAKMDTASMLDEAIRYTKFLKRQVRILQPHSQIGAPMANPSYLCYYHNSQP

SEQ ID NO: 40 - protein, 270 aa, Glycine max
MDTNTSTLFTNVNSTWNLEKMETNEQQQQHDDHSIILQVQDPMGSGIWPINNYQNLLQMHQTPNTTTSSTVIVPPSSSSGFLGDILGVHHNLEEDEEPEEELGAMKEMMYKIAAMQPVDIDPATIRKPKRRNVRISDDPQSVAARHRRERISEKIRILQRLVPGGTKMDTASMLDEAIRYVKFLKRQIRLLQSIPQPSRQPPQCIGVASTTPHASTLLLAPSSDWPFAPNVLPRSTAVSASMDMSAGLGFDGHAHACDGSSSFNHHEVIS

SEQ ID NO: 41 - protein, 270 aa, Oryza sativa; identical to SEQ ID NO: 40

SEQ ID NO: 42 - protein, 283 aa, Solanum lycopersicum
MDINHINKLTTSTWDPTMSNMDNQQVFRDQQQQQQPCLSSIPNDHIYHEHHHHQQQFHFEHNPIWPSFPLQNPQHHHLPSSSTQQQQQQQEEVVVVPFDHVLNNHVQTLIEDQEHDDQDEDEEEEEELGAMKEMMFKIASMQPVDIDPSTIRKPKRRNVRISNDPQSVAARLRRERISEKIRILQRLVPGGTKMDTASMLDEAIRYVKFLKRQIRQLQSSNHNLPPAQIPVSSCPNNENWANNIVTPSTKGLILGSSSSTTTNNVTTFVGNTTLDPPYEVIGN

SEQ ID NO: 43 - DNA, 108 bp, synthetic construct
  variation 54: note = n = a, c, t or g
  source 1..108: mol_type = other DNA
atccttgtctgtgttggtggtaatcagcattgggtttaggcctaaaccattcangactgctaaagtctgcattatccattgtatgatcaaataagcgctgagttttac

SEQ ID NO: 44 - DNA, 108 bp, synthetic construct
  source 1..108: mol_type = other DNA
atccttgtctgtgttggtggtaatnncatttgggcntangccnntcccgancgcgacngccagcgtcagccntagcnngngcangaacncncngacgntgantttnan

SEQ ID NO: 45 - DNA, 107 bp, synthetic construct
  source 1..107: mol_type = other DNA
atccttgtctgtgttggtgataangcnttgaggncaagcccantccagncngganngntnngnnctnncntatgcatngtnngatnannnntgagctganntttana

SEQ ID NO: 46 - DNA, 108 bp, Thlaspi arvense
  source 1..108: mol_type = genomic DNA
atccttgtctgtgttggtggtaatcgcattgggtttaggcctaaaccattcatgactgctaaagtctgcattatccattgtatgatcaaataagcgctgagttttaca

SEQ ID NO: 47 - protein, 438 aa, Arabidopsis thaliana [sequence omitted]

SEQ ID NO: 48 - protein, 452 aa, Glycine max
METGGGRDSGMSSETINSSTQRTSMSNESVCSTSFSRLSFDLPPPSSSPETLFVKPHRSSDFAYSAILRRKSALTFRDFHLLRRIGAGDIGTVYLCRLRHDAGDEDDDEDPCFYAMKVVDKEAVALKKKAQRAEMERKILKMVDHPFLPTLYAEFEASNFSCIVMEYCSGGDLHSLQHNHPNNRFSLSSARFYAAEVLVALEYLHMLGIIYRDLKPENVLVRSDGHIMLSDFDLSLCSDAIPAVESPDCSLDPAFAPALRYTRQYSTPFSCLSNRVFRSRKVQTLQPNRLFVAEPVGARSCSFVGTHEYVSPEVASGNSHGNAVDWWSFGIFIYEMVYGRTPFAGSSNEATLRSIIKKPLAFPTSTPSSTLEMHARDLISGLLNKDPNRRLGSKRGSADVKKHPFFAGLNLALIRTVTPPEVPSLRRHKTTPFYYPANVNNSRQQLTAFDYF

SEQ ID NO: 49 - protein, 484 aa, Oryza sativa
MVAAVRAPVKPEMVELSPAAMERYSSDADTTAPNSSLSSAASSTGSLARCSSLSRLSFDCSPSAAVAAAATSCSPPRASVLLRPHRSGDVAWAAIRAASTTSAAPLGPRDFKLVRRIGGGDIGTVYLCRLRSSPERESPCMYAMKVVDRRAVARKQKLGRAAAEKRILRQLDHPFLPTLFADFDATPHFSCAVMEFCPGGDLHSLRHRMPSRRFPLPSARFYAAEVLLAIEYLHMMGIVYRDLKPENVLIRADGHIMLTDFDLSLQSTTSPSLDGDTDTDDEASGGASCFPDHLLRFKRRRNAVAAPRPRFVAEPVDARSCSFVGTHEYVAPEVASGGAHGAAVDWWAYGVFLYELIYGRTPFAGATNEATLRNIVRRPLAFPSGSGSCGPADADARDLIARLLAKDPAARLGSRRGAADVKSHPFFKSLNLALLRSSRPPVVPGAGAGAAPLHRSQSCKAAPTTPPPPTTTKPANATARFDLF

SEQ ID NO: 50 - protein, 462 aa, Solanum lycopersicum
MATTNRDESDKDSTASSSITMPESSRRSWMSSTNLSSFSSRRSSISLCNENPYFSNSHKPHKSNQISWELIRRIRVESGQIKLEHFRLLRRVGGGDIGSVYLCEIRNPVVGLPQCFYAMKVVDREAVEIRKKLQRGEMEKEILGIIDHPFLPTLYAQFEASHYSCLVMEYCPGGDLHAVRQRQPGKRFSISSAKFYAAEILLALEYLHMMGIVYRDLKPENVLVRSDGHIMLSDFDLSFKCDEVVPTLVKSKTTKSIAKTPRNSYCAMPIQPVLSCFLSQKTEQNHENQEEDQEIVAEPINARSKSFVGTHEYLAPEVISGQGHGSAVDWWTLGVFLYELIFGTTPFKGENNEKTLVNILKKPLTFPRIAISSSKEYEEMVKVQDLISRLLVKNPKKRIGSLQGSVEIKKHEFFKGVNWALIRSIKPPQVPNDLVKMRGVVPKLSKKQREEPYQIPQYFDYF

SEQ ID NO: 51 - DNA, 20 bp, synthetic construct
  source 1..20: mol_type = other DNA
gggcaatgttattacctccg

SEQ ID NO: 52 - DNA, 20 bp, synthetic construct
  source 1..20: mol_type = other DNA
ggctctatgacagaccaatc

SEQ ID NO: 53 - DNA, 24 bp, synthetic construct
  source 1..24: mol_type = other DNA
attgtgcgattaccaccaacacag

SEQ ID NO: 54 - DNA, 24 bp, synthetic construct
  source 1..24: mol_type = other DNA
aaacctgtgttggtggtaatcgca

SEQ ID NO: 55 - DNA, 30 bp, synthetic construct
  source 1..30: mol_type = other DNA
aggagctaaacatcgaaattcgttgaagag

SEQ ID NO: 56 - DNA, 30 bp, synthetic construct
  source 1..30: mol_type = other DNA
tgtcgcagatactagaggaacatcacatca

SEQ ID NO: 57 - DNA, 20 bp, synthetic construct
  source 1..20: mol_type = other DNA
gatggatatggtcatgaaga

SEQ ID NO: 58 - DNA, 20 bp, synthetic construct
  source 1..20: mol_type = other DNA
tcttcatgaccatatccatc

SEQ ID NO: 59 - DNA, 50 bp, Thlaspi arvense
  source 1..50: mol_type = genomic DNA
cttgtctgtgttggtggtaatcgcattgggtttaggcctaaaccattcat

SEQ ID NO: 60 - DNA, 50 bp, synthetic construct
  source 1..50: mol_type = other DNA
cttgtctgtgttggtggtaatcacattgggtttaggcctaaaccattcat

SEQ ID NO: 61 - DNA, 44 bp, synthetic construct
  source 1..44: mol_type = other DNA
cttgtctgtgttggtggtaatgggtttaggcctaaaccattcat

SEQ ID NO: 62 - DNA, 41 bp, synthetic construct
  source 1..41: mol_type = other DNA
cttgtctgtgttggtggtgggtttaggcctaaaccattcat

SEQ ID NO: 63 - DNA, 45 bp, synthetic construct
  source 1..45: mol_type = other DNA
cttgtctgtgttggtggtaattgggtttaggcctaaaccattcat

SEQ ID NO: 64 - DNA, 45 bp, synthetic construct
  source 1..45: mol_type = other DNA
cttgtctgtgttggtggtgcatgggtttaggcctaaaccattcat

SEQ ID NO: 65 - DNA, 47 bp, synthetic construct
  source 1..47: mol_type = other DNA
cttgtctgtgttggtggtaatcatgggtttaggcctaaaccattcat

SEQ ID NO: 66 - DNA, 49 bp, synthetic construct
  source 1..49: mol_type = other DNA
cttgtctgtgttggtgattaagcattgggtttaggcctaaaccattcat

SEQ ID NO: 67 - DNA, 50 bp, synthetic construct
  source 1..50: mol_type = other DNA
cttgtctgtgttggtggtaatcagcattgggtttaggcctaaaccattca

SEQ ID NO: 68 - DNA, 50 bp, synthetic construct
  source 1..50: mol_type = other DNA
cttgtctgtgttggtggtaatctgcattgggtttaggcctaaaccattca

SEQ ID NO: 69 - DNA, 591 bp, Thlaspi arvense
  source 1..591: mol_type = genomic DNA
atgtttggctcaaaagcagatccacccataaccccaatagtcatgatggagcctcaacctcatcatctcctcatgaattggaacaaacctaatgatctcatcacacaagaatacccctttctccacgatcctcatctcatgatagatccacctcccgaaaccctaagtcatttccagcccccgccgacacttttctccggtcacggaggggaggaagaagaagaagaagataatgaagaggaagagatggatgcgatgaaggagatgcagtacacgatcgctgccatgcagcccgtggacatcgatccagccaccgttcctaaaccgaaccgccgtaacgtaagggtaagcgacgacactcagacggtggtggctcgtcggcgtcgagaaaagataagcgagaagatccgaatattgaagaggatggtgccaggcggtgcgaagatggacacagcctccatgctcgacgaagccatccgttataccaagttcttgaaacggcaggtgaagcttcttcagcctcactctcagcttggagctcctatgtctgacccctcttgcctttgttattaccacaactcccaaacctaa

SEQ ID NO: 70 - protein, 196 aa, Thlaspi arvense
MFGSKADPPITPIVMMEPQPHHLLMNWNKPNDLITQEYPFLHDPHLMIDPPPETLSHFQPPPTLFSGHGGEEEEEEDNEEEEMDAMKEMQYTIAAMQPVDIDPATVPKPNRRNVRVSDDTQTVVARRRREKISEKIRILKRMVPGGAKMDTASMLDEAIRYTKFLKRQVKLLQPHSQLGAPMSDPSCLCYYHNSQT

(19) United States
(12) Patent Application Publication
(10) Pub. No.: US 2024/0065193 A1
(43) Pub. Date: Feb. 29, 2024

(54) OILSEED PLANTS HAVING REDUCED POD SHATTER

(71) Applicants: Regents of the University of Minnesota, Minneapolis, MN (US); The Board of Trustees of Illinois State University, Normal, IL (US)

(72) Inventors: Michael David Marks, Roseville, MN (US); John C. Sedbrook, Bloomington, IL (US); Ratan Chopra, Minneapolis, MN (US); Maliheh Esfahanian, Bloomington, IL (US)

(21) Appl. No.: 18/137,263

(22) Filed: Apr. 20, 2023

Related U.S. Application Data
(62) Division of application No. 16/104,478, filed on Aug. 17, 2018, now Pat. No. 11,707,029.
(60) Provisional application No. 62/547,684, filed on Aug. 18, 2017.

(51) Int. Cl. A01H 6/20; C07K 14/415; C12N 9/24; C12N 15/82

(52) U.S. Cl. CPC A01H 6/20; C07K 14/415; C12N 9/2402; C12N 15/8213; C12N 15/8266; C12Y 302/01015; C12N 9/24

(57) ABSTRACT

This document relates to materials and methods for domesticating oilseed (e.g., pennycress) plants. For example, oilseed plants having reduced seedpod shatter, as well as materials and methods for making and using oilseed plants having reduced seedpod shatter are provided.

### CROSS-REFERENCE TO RELATED APPLICATIONS

This application is a Divisional of, and claims the benefit of priority, U.S. application Ser. No. 16/104,478 filed on Aug. 17, 2018, which also claims the benefit of priority of U.S. Application Ser. No. 62/547,684, filed on Aug. 18, 2017. The disclosures of the prior applications are considered part of (and are incorporated by reference in) the disclosure of this application.

### STATEMENT REGARDING FEDERAL FUNDING

This invention was made with government support under 2014-67009-22305 awarded by the United States Department of Agriculture. The government has certain rights in the invention.

### SEQUENCE LISTING

This application contains a Sequence Listing that has been submitted electronically as an XML file named 09531-040002_SL_ST26.xml. The XML file, created on Apr. 19, 2023, is 118,061 bytes in size. The material in the XML file is hereby incorporated by reference in its entirety.

### BACKGROUND

### 1. Technical Field

This document relates to materials and methods for domesticating oilseed (e.g., pennycress) plants. For example, this document provides oilseed plants having reduced seedpod shatter, as well as materials and methods for making and using oilseed plants having reduced seedpod shatter.

### 2. Background Information

Across the Upper Midwestern USA tens of millions of acres of farmland are rotated between Zea mays L. and Glycine max (L.) Merr. Only 2% of this region is typically protected by a cover crop in the fallow period between autumn harvest and the following spring planting. This leaves farmlands across the Midwest vulnerable to nutrient leaching and soil erosion, threatening the sustainability of current farming practices and the health of rural populations. Cover crops such as winter rye (Secale cereale L.) that can be grown during the fallow period can greatly reduce nutrient leaching; however, rye provides no economic return.

### SUMMARY

This document provides materials and methods for domesticating oilseed (e.g., pennycress) plants. For example, this document provides domesticated oilseed plants having reduced seedpod shatter, as compared to corresponding wild type oilseed plants. This document also provides materials and methods for making and/or using the domesticated oilseed plants described herein (e.g., having reduced seedpod shatter).

As demonstrated herein, loss-of-function modifications in the pennycress SPATULA (SPT), ALCATRAZ (ALC), INDEHISCENT (IND), SHATTERPROOF (SHP; e.g., SHP1 and SHP2), PINOID (PID), and DZ POLYGALACTURONASE (ADPG; e.g., ADPG1 and ADPG2) genes resulted in reduced seedpod shatter as compared to corresponding wild type pennycress plants. Domesticated oilseed plants having reduced seedpod shatter can have increased yield (e.g., increased harvestable plant yield and/or longer harvest-time windows) as compared to corresponding wild type pennycress plants.

Having the ability to design oilseed (e.g., pennycress) plants having reduced seedpod shatter provides a unique and unrealized opportunity to improve the sustainability of current farming practices. For example, oilseed plants provided herein can be grown in the interval between corn harvest and soybean establishment the following spring in the Midwestern United States as well as in other regions and cropping systems, thereby maximizing potential production from land already in use. Oil from the seeds of oilseed plants provided herein can be used as edible oil or used for the production of biodiesel, jet fuel, and other bioproducts. In addition, oilseed plants provided herein can provide ecosystem services such as reducing nutrient leaching and soil erosion and providing food for pollinators. Thus, pennycress plants described here have the potential to reduce nutrient leaching while providing farmers with an oilseed cash crop.

In general, one aspect of this document features an oilseed plant having reduced seedpod shatter (e.g., as compared to a corresponding wild type oilseed plant), where the oilseed plant includes a modification in one or more genes encoding a polypeptide and/or a microRNA involved in seedpod shatter. The oilseed plant can be a pennycress plant. The oilseed plant can include seedpods that require about 20 grams to about 120 grams pulling force to shatter. The oilseed plant can include seedpods that are resistant to shatter under less than about 30 grams force. The modification can be a loss-of-function modification. In some cases, the gene can be a SPT gene. The modified SPT gene can include a single base-pair substitution. The single base-pair substitution can be a C to T substitution at nucleotide residue 157. The modified SPT gene can include the sequence set forth in SEQ ID NO:3. The modified SPT gene can encode a modified SPT polypeptide. The modified SPT polypeptide can include the sequence set forth in SEQ ID NO:4. In some cases, the gene can be a ALC gene. The modified ALC gene can include a 10 base pair deletion and a 4 base pair insertion. The oilseed plant of claim 13, wherein said 10 base pair deletion includes deletion of residues 827 to 836. The 4 base pair insertion can be a TCTC insertion following nucleotide residue 827. The modified ALC gene can include the sequence set forth in SEQ ID NO:7. The modified ALC gene can encode a modified ALC polypeptide. The modified ALC polypeptide can be as set forth in SEQ ID NO:8.

In another aspect, this document features an oilseed plant having reduced seedpod shatter (e.g., as compared to a corresponding wild type oilseed plant). The oilseed plant can have a modification in one or more genes encoding a polypeptide and/or a microRNA involved in seedpod shatter. The gene encoding a polypeptide and/or a microRNA involved in seedpod shatter can be selected from the group consisting of SPT, ALC, IND, RPL, SHP1, SHP2, FUL, NST1, NST3, PID, ADPG1, and/or ADPG2. The modified gene can encode a modified polypeptide and/or a modified microRNA. The oilseed plant can be a pennycress plant. The oilseed plant can have seedpods that require about 20 grams to about 120 grams pulling force to shatter. The oilseed plant can have seedpods that are resistant to shatter under less than about 30 grams force. When the gene encoding a polypeptide involved in seedpod shatter is a SPT gene, the modified SPT gene can encode a modified SPT polypeptide. The modified SPT gene can include a C to T substitution at nucleotide residue 157 as set forth in SEQ ID NO:3, and the modified SPT polypeptide can include the amino acid sequence set forth in SEQ ID NO:4. When the gene encoding a polypeptide involved in seedpod shatter is a ALC gene, the modified ALC gene can encode a modified ALC polypeptide. The modified ALC gene can include a 10 base pair deletion of residues 827 to 836 and a TCTC insertion following nucleotide residue 827 as set forth in SEQ ID NO:7, and wherein said modified ALC polypeptide can include the amino acid sequence set forth in SEQ ID NO:8. When the gene encoding a polypeptide involved in seedpod shatter is a IND gene, the modified IND gene can encode a modified IND polypeptide. The modified IND gene can include a C to T substitution at nucleotide residue 247 as set forth in SEQ ID NO:11, and the modified IND polypeptide can include the amino acid sequence set forth in SEQ ID NO:12. The modified IND gene can include a G to A substitution at nucleotide residue 260 as set forth in SEQ ID NO:13, and the modified IND polypeptide can include the amino acid sequence set forth in SEQ ID NO:14. The modified IND gene can include a G to A substitution at nucleotide residue 301 as set forth in SEQ ID NO:15, and the modified IND polypeptide can include the amino acid sequence set forth in SEQ ID NO:16. When the gene encoding a polypeptide involved in seedpod shatter is a SHP1 gene, the modified SHP1 gene can encode a modified SHP1 polypeptide. The modified SHP1 gene can include a G to A substitution at nucleotide residue 17 as set forth in SEQ ID NO:19, and the modified SHP1 polypeptide can include the amino acid sequence set forth in SEQ ID NO:20. When the gene encoding a polypeptide involved in seedpod shatter is a SHP2 gene, the modified SHP2 gene can encode a modified SHP2 polypeptide. The modified SHP2 gene can include a G to A substitution at nucleotide residue 175 as set forth in SEQ ID NO:23, and the modified SHP2 polypeptide can include the amino acid sequence set forth in SEQ ID NO:24. When the gene encoding a polypeptide involved in seedpod shatter is a PID gene, the modified PID gene encodes a modified PID polypeptide. The modified PID gene can include a G to A substitution at nucleotide residue 470 as set forth in SEQ ID NO:27, and the modified PID polypeptide can include the amino acid sequence set forth in SEQ ID NO:28. When the gene encoding a polypeptide involved in seedpod shatter is a ADPG1 gene, the modified ADPG1 gene can encode a modified ADPG1 polypeptide. The modified ADPG1 gene can include single base pair deletion of residue 104 as set forth in SEQ ID NO:31, and the modified ADPG1 polypeptide can include the amino acid sequence set forth in SEQ ID NO:32. The modified ADPG1 gene can include a 4 base pair deletion of residues 101 to 104 as set forth in SEQ ID NO:33, and the modified ADPG1 polypeptide can include the amino acid sequence set forth in SEQ ID NO:34.

In another aspect, this document features a seed produced by an oilseed plant having reduced seedpod shatter (e.g., as compared to a corresponding wild type oilseed plant), where the oilseed plant includes a modification in one or more genes encoding a polypeptide and/or a microRNA involved in seedpod shatter.

In another aspect, this document features a method for generating an oilseed plant having reduced seedpod shatter (e.g., as compared to a corresponding wild type oilseed plant). The method includes, or consists essentially of, modifying a gene in the oilseed plant genome, where the gene encodes a polypeptide and/or a microRNA involved in seedpod shatter, and where the modification is effective to reduce pod shatter in the plant. The modifying can include site-specific editing. The oilseed plant can be a pennycress plant. The oilseed plant can include seedpods that require about 20 grams to about 120 grams pulling force to shatter. The modification can be a loss-of-function modification. In some cases, the gene can be a SPT gene. The modified SPT gene can include a single base-pair substitution (e.g., a C to T substitution) at nucleotide residue 157. The modified SPT gene can include the sequence set forth in SEQ ID NO:3. The modified SPT gene can encode a modified SPT polypeptide. The modified SPT polypeptide can include the sequence set forth in SEQ ID NO:4. In some cases, the gene can be a ALC gene. The modified ALC gene can include a 10 base pair deletion of residues 827 to 836, and a 4 base pair TCTC insertion following nucleotide residue 827. The modified ALC gene can include the sequence set forth in SEQ ID NO:7. The modified ALC gene can encode a modified ALC polypeptide. The modified ALC polypeptide can include the sequence set forth in SEQ ID NO:8.

In another aspect, this document features a method for generating an oilseed plant having reduced seedpod shatter (e.g., as compared to a corresponding wild type oilseed plant). The method includes, or consists essentially of, introducing into an oilseed plant cell a nuclease and a guide sequence, where the guide sequence includes a nucleic acid sequence specific to the ALC gene; selecting an oilseed plant cell having reduced seedpod shatter as compared to a wild oilseed plant; and regenerating an oilseed plant having reduced seedpod shatter from the selected oilseed plant cell. The oilseed plant can be a pennycress plant. The nuclease can be a clustered regularly interspaced short palindromic repeats (CRISPR) associated system (Cas) nuclease. The Cas nuclease can be a Cas9 nuclease. The Cas9 nuclease can be a Staphylococcus aureus Cas9. The guide sequence can include SEQ ID NO:53.

In another aspect, this document features a method for generating an oilseed plant having reduced seedpod shatter (e.g., as compared to a corresponding wild type oilseed plant). The method includes, or consists essentially of, modifying one or more genes in the oilseed plant genome, where the gene encodes a polypeptide and/or a microRNA involved in seedpod shatter. The gene that encodes a polypeptide involved in seedpod shatter can be SPT, ALC, IND, RPL, SHP1, SHP2, FUL, NST1, NST3, PID, ADPG1, and/or ADPG2. The modified gene can encode a modified polypeptide. The oilseed plant can be a pennycress plant. The modifying can include site-specific editing. The site-specific editing can include introducing a guide sequence and a nuclease into an oilseed plant cell, where the guide sequence includes a nucleic acid sequence specific to a gene encoding a polypeptide involved in seedpod shatter, and where the nuclease is a Cas nuclease. The modifying can include mutagenesis. The mutagenesis can include introducing a mutagen into an oilseed plant cell. The mutagen can include ethyl methane sulphonate (EMS). The method also can include selecting an oilseed plant cell having reduced seedpod shatter as compared to a wild oilseed plant; and regenerating an oilseed plant having reduced seedpod shatter from the selected oilseed plant cell.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains a schematic of the polypeptides controlling the cascade of events leading to formation and a weakening of the layer of cells in the Arabidopsis seedpod referred to as the valve margin. This region is highlighted in the corresponding regions of pennycress pods. The weakening allows the pods to readily split as shown. Also shown are the nature of various mutant alleles for most of the genes in the pathway (mutant alleles (purple), (amino acid changes (blue), position of changes in the peptides (red)).

FIGS. 2A-2D show exemplary SPT sequences. A) A wild type (WT) pennycress SPT nucleotide sequence (SEQ ID NO:1) with lowercase letters indicating introns. B) A WT pennycress SPT polypeptide sequence (SEQ ID NO:2). C) A modified pennycress SPT nucleotide sequence having a C to T substitution at nucleotide residue 157 (SEQ ID NO:3) with the bold, italic T indicating the substitution and the lower case letters indicating introns. D) A modified pennycress SPT polypeptide sequence having a R to W substitution at amino acid residue 53 (SEQ ID NO:4) with the bold, italic W indicating the substitution.

FIGS. 3A-3E show exemplary ALC sequences. A) A WT pennycress ALC nucleotide sequence (SEQ ID NO:5) with lower case letters indicating introns. A protospacer location and PAM site that can be targeted by, for example, CRISPR-SaCas9 gRNA are underlined and in bold (protospacer) or italicized (PAM site). B) A WT pennycress ALC polypeptide sequence (SEQ ID NO:6). C) A modified pennycress ALC nucleotide sequence having a 10 base pair deletion of residues 827 to 836, and a 4 base pair TCTC insertion following nucleotide residue 827 (SEQ ID NO:7) with the bold, italic TCTC indicating the insertion and the letters indicating the deletion. D) A modified pennycress ALC polypeptide sequence having a deletion of asparagine-proline-methionine-arginine (NPMR) at residues 160-163, and an insertion of isoleucine-serine (IS) at residues 160-161 (SEQ ID NO:8) with the bold, italic IS indicating the substituted amino acids. E) Additional exemplary modified pennycress ALC nucleotide sequences having deletions, insertions, and/or base substitutions induced in the pennycress ALC gene at the CRISPR-SaCas9 gRNA site (mutations highlighted in red) using CRISPR-Cas9 methodology. Sequences shown include, from top to bottom, a fragment of a wild type ALC nucleotide sequence (SEQ ID NO:59), a G to A transition (SEQ ID NO:60), a 6 base pair deletion found in the alc-1 mutant (SEQ ID NO:61), a 9 base pair deletion (SEQ ID NO:62), a 4 base pair deletion in the alc-2 mutant (SEQ ID NO:63), a 4 base pair deletion (SEQ ID NO:64), a 2 base pair deletion in the alc-3 mutant (SEQ ID NO:65), a 6 base pair deletion plus a 5 base pair insertion (SEQ ID NO:66), a 1 base pair insertion in the alc-4 mutant (SEQ ID NO:67), and a 1 base pair insertion (SEQ ID NO:68).

FIGS. 4A-4H show exemplary IND sequences. A) WT pennycress IND nucleotide sequences (SEQ ID NO:9 and SEQ ID NO:69). B) WT pennycress IND polypeptides sequence (SEQ ID NO:10 and SEQ ID NO:70). C) A modified pennycress IND nucleotide sequence having a C to T substitution at nucleotide residue 247 (SEQ ID NO:11) with the bold, italic T indicating the substitution. D) A modified pennycress IND polypeptide sequence having a P to S substitution at amino acid residue 83 (SEQ ID NO:12) with the bold, italic S indicating the substitution. E) A modified pennycress IND nucleotide sequence having a G to A substitution at nucleotide residue 260 (SEQ ID NO:13) with the bold, italic A indicating the substitution. F) A modified pennycress IND polypeptide sequence having a R to H substitution at amino acid residue 87 (SEQ ID NO:14) with the bold, italic H indicating the substitution. G) A modified pennycress IND nucleotide sequence having a G to A substitution at nucleotide residue 301 (SEQ ID NO:15) with the bold, italic G indicating the substitution. H) A modified pennycress IND polypeptide sequence having a A to T substitution at amino acid residue 101 (SEQ ID NO:16) with the bold, italic T indicating the substitution. Residue numbers in FIGS. 4C-4H are with reference to SEQ ID NO:9 or SEQ ID NO:10.

FIGS. 5A-5D show exemplary SHP1 sequences. A) A WT pennycress SHP1 nucleotide sequence (SEQ ID NO:17). B) A WT pennycress SHP1 polypeptide sequence (SEQ ID NO:18). C) A modified pennycress SHP1 nucleotide sequence having a G to A substitution at nucleotide residue 17 (SEQ ID NO:19) with the bold, italic A indicating the substitution. D) A modified pennycress SHP1 polypeptide sequence having a S to N substitution at amino acid residue 6 (SEQ ID NO:20) with the bold, italic N indicating the substitution.

FIGS. 6A-6D show exemplary SHP2 sequences. A) A WT pennycress SHP2 nucleotide sequence (SEQ ID NO:21). B) A WT pennycress SHP2 polypeptide sequence (SEQ ID NO:22). C) A modified pennycress SHP2 nucleotide sequence having a G to A substitution at nucleotide residue 175 (SEQ ID NO:23) with the bold, italic A indicating the substitution. D) A modified pennycress SHP2 polypeptide sequence having an A to T substitution at amino acid residue 59 (SEQ ID NO:24) with the bold, italic T indicating the substitution.

FIGS. 7A-7D show exemplary PID sequences. A) A WT pennycress PID nucleotide sequence (SEQ ID NO:25). B) A WT pennycress PID polypeptide sequence (SEQ ID NO:26). C) A modified pennycress PID nucleotide sequence having a G to A substitution at nucleotide residue 470 (SEQ ID NO:27) with the bold, italic A indicating the substitution. D) A modified pennycress PID polypeptide sequence having a C to Y substitution at amino acid residue 157 (SEQ ID NO:28) with the bold, italic Y indicating the substitution.

FIGS. 8A-8F show exemplary ADPG1 sequences. A) A WT pennycress ADPG1 nucleotide sequence (SEQ ID NO:29). A protospacer location and PAM site that can be targeted by, for example, CRISPR-SaCas9 gRNA are underlined and in bold (protospacer) or italicized (PAM site). B) A WT pennycress ADPG1 polypeptide sequence (SEQ ID NO:30). C) A modified pennycress ADPG1 nucleotide sequence having a single base pair deletion of residue 104 (SEQ ID NO:31) with the letter indicating the deletion. The aberrant amino acid sequence leading up to the stop codon is highlighted in yellow. D) A modified pennycress ADPG1 polypeptide sequence having a truncation (SEQ ID NO:32) with the aberrant amino acid residues (highlighted in bold; resulting from a frameshift) leading up to the stop codon (highlighted in bold in SEQ ID NO:31). E) A modified pennycress ADPG1 nucleotide sequence having a 4 base pair deletion of residues 101 to 104 (SEQ ID NO:33) with the letters indicating the deletion. F) A modified pennycress ADPG1 polypeptide sequence having a truncation (SEQ ID NO:34) with the aberrant amino acid residues (highlighted in bold; resulting from a frameshift) leading up to the stop codon (highlighted in bold in SEQ ID NO:33).

FIG. 9 contains a sequence alignment of SPT polypeptides from Arabidopsis thaliana (At_SPT; SEQ ID NO:35), Thlaspi arvense (Ta_SPT; SEQ ID NO:2), a modified Thlaspi arvense (Ta_spt-1; mutant strain A7-129; SEQ ID NO:4), Glycine max (Gm_SPT; SEQ ID NO:36), Oryza sativa (Os_SPT; SEQ ID NO:37), and Solanum lycopersicum (Sl_SPT; SEQ ID NO:38).

FIGS. 10A-10G show an analysis of seedpod shatter in pennycress plants. A-C) Images showing field grown mature pods from wild type pennycress plants (A), a mutant pennycress plant having a modified SPT gene (line MNA7-129) (B), and a mutant pennycress plant having a modified SPT gene (line MNA7-236)(C). D) A gene model for a mutant pennycress plant having a modified SPT gene (line MNA7-129). E) A graph showing losses due to shatter induced by environmental conditions or by mechanical harvest machinery. F) A graph showing gram-force needed to break open wild type (line MN 106) and mutant (lines MNA7-129 and MNA7-236) seedpods. G) Greater force is required to break open the seedpods of the mutant pennycress plants having a modified SPT gene.

FIGS. 11A-11E show an analysis of seedpod shatter in pennycress plants. A-C) Microscopic images of seedpods from a wild type pennycress plant (A), mutant pennycress plant having a modified ALCATRAZ gene (line ME5) (B), and seedpods from a mutant pennycress plant having a modified ALCATRAZ gene (line alc-4) (C). D) A graph with averages of the amounts of pulling force necessary to break the pods open. Asterisks represent significant differences compared to wild type as determined by Student's t-test. Bars represent standard deviations; n=12. E) Greater force is required to break open the seedpods from a mutant pennycress plant having a modified ALCATRAZ gene (lines alc-1, alc-2, alc-3, and alc-4).

FIG. 12 contains a sequence alignment of IND polypeptides from Arabidopsis thaliana (At_SPT; SEQ ID NO:39), Thlaspi arvense (Ta_SPT; SEQ ID NO:10), a modified Thlaspi arvense (Ta_ind-1; mutant strain E5-552; SEQ ID NO:12), a modified Thlaspi arvense (Ta_ind-2; mutant strain E5-550; SEQ ID NO:14), a modified Thlaspi arvense (Ta_ind-3; mutant strain Spring32_NS; SEQ ID NO:16), Glycine max (Gm_IND; SEQ ID NO:40), Oryza sativa (Os_IND; SEQ ID NO:41), and Solanum lycopersicum (Sl_IND; SEQ ID NO:42).

FIGS. 13A-13F show an analysis of seedpod shatter in pennycress plants. A) Images showing growth chamber-grown mature pods for wild type and rps1-1 mutant (line E42; also referred to as ind-3) pennycress plants. rps1-1 plants grew like wild type. B) Images showing seedpods from wild type and rps1-1 mutant (line E42; also referred to as ind-3) pennycress plants. rps1-1 plants had reduced seedpod shatter compared to wild-type pods. C) Microscopic images of pennycress seedpods from a wild type pennycress plant D) Microscopic images of seedpods from a ind-3 (rps1-1) mutant (line E42) and wild type. rps1-1 pods show no dehiscence zone separation. E) A graph with averages of the amounts of pulling force necessary to break the pods open. Seedpods from a mutant pennycress plant having a modified IND gene (ind-3; also referred to as rps1-1) often tore open within a valve instead of at the dehiscence zone. Asterisks represent significant differences compared to wild type as determined by Student's t-test. Bars represent standard deviations; n=12. F) Greater force is required to break open the seedpods from a mutant pennycress plant having a modified IND gene (lines ind-1 and ind-2). Bars represent standard deviations. Letters that are different represent significant differences based on ANOVA.

FIGS. 14A-14B show an exemplary mechanism for IND loss of function in seedpods from a mutant pennycress plant having a modified IND gene (line ind-3). A) Protein modeling of IND was performed using SWISS-MODEL (swissmodel.expasy.org/). B) The alanine to threonine amino acid substitution in ind-3 likely affects protein binding to the DNA. IND encodes a transcription factor that regulates expression of seedpod shatter-related genes.

FIG. 15 shows an image of an agarose gel containing electrophoresed ALCATRAZ-gene PCR products, amplified from six CRISPR-Cas9 ALCATRAZ T2-generation plants and from wild type; those products were digested with the T7 endonuclease enzyme. T7 endonuclease cuts DNA at sites where base-pair mismatches are present. Therefore, the agarose gel data indicate plant lines ME3, ME5, and ME6 likely have CRISPR-Cas9-induced edits that caused DNA base-pair mismatches. Also shown are DNA sequence chromatograms obtained from sequencing the PCR products derived from plant lines ME3 (SEQ ID NO:43), ME5 (SEQ ID NO:44), ME6 (SEQ ID NO:45), and wild type (SEQ ID NO:46); arrows indicate locations where genome edits had occurred near the PAM site (different mutations were observed on each chromosome pair) resulting in likely loss-of-function mutations including frameshift mutations and the observed reduced seedpod shatter phenotypes.

FIG. 16 contains microscopic images of toluidine blue-stained cross sections of a wild-type seedpod (left panel) and a seedpod from a pennycress plant having a modified IND gene (line ind-3 (rps1-1) right panel)) showing that the separation layer of cells did not form in the rps1-1 pod dehiscence zone.

FIG. 17 contains a sequence alignment of PID polypeptides from Thlaspi arvense (Ta_PID; SEQ ID NO:26), a modified Thlaspi arvense (Ta_pid-1; mutant strain A7-236; SEQ ID NO:28), Arabidopsis thaliana (At_PID; SEQ ID NO:47), Glycine max (Gm_IND; SEQ ID NO:48), Oryza sativa (Os_IND; SEQ ID NO:49), and Solanum lycopersicum (Sl_IND; SEQ ID NO:50).

FIGS. 18A-18C show an analysis of seedpod shatter in pennycress plants. A) An image showing field grown mature pods for a wild type pennycress plant. B) An image showing field grown mature pods for a representative mutant pennycress plant having a modified PID gene (line pid-1). The pennycress plant having a modified PID gene shows few shattered pods as highlighted by arrows in the wild type image. C) Greater force is required to break open the seedpods from a mutant pennycress plant having a modified PID gene (line pid-1). Bars represent standard deviations. Letters that are different represent significant differences based on ANOVA.

FIGS. 19A-19C show an analysis of seedpod shatter in pennycress plants. A) A microscopic images of seedpods from a wild type pennycress plant. B) A microscopic images of seedpods from a mutant pennycress plant having a modified ADPG1 gene (line adgp1-2). C) Greater force is required to break open the seedpods from a mutant pennycress plant having a modified ADPG1 gene (lines adpg1-1 and adpg1-2). Asterisks represent significant differences compared to wild type as determined by Student's t-test. Bars represent standard deviations; n=30.

FIG. 20 is a graph showing the percent of non-shattered pods on plants over time. The percent of pods remaining on plants was measured at one-week intervals after pod maturation. All three reps of wild type showed nearly a 100% loss after three weeks whereas ind-2, spt-1, and pid-1 lines retained 60-90% of their seedpods during the same timeframe. All mutant phenotypes at the later time points are significantly different from wild type based on an ANOVA statistical analysis. Bars represent standard deviations.

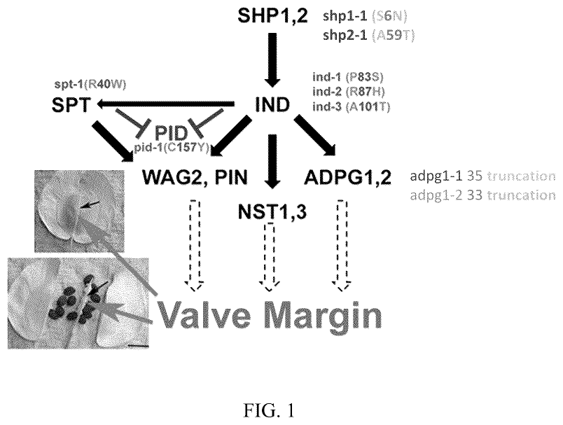

### DETAILED DESCRIPTION

This document provides oilseed (e.g., pennycress) plants having reduced seedpod shatter. In some cases, this document provides oilseed plants having reduced expression levels of one or more polypeptides involved in seedpod shatter (e.g., as compared to corresponding wild type plants). For example, an oilseed plant having reduced seedpod shatter can have a one or more modifications in one or more genes encoding a polypeptide involved in seedpod shatter where the modification(s) are effective to reduce polypeptide expression. In some cases, this document provides oilseed plants having reduced function of one or more polypeptides involved in seedpod shatter (e.g., as compared to corresponding wild type plants). For example, an oilseed plant having reduced seedpod shatter can have one or more modifications in a polypeptide involved in seedpod shatter where the modification(s) are effective to reduce polypeptide function.

This document also provides materials and methods for making and/or using the oilseed plants described herein (e.g., oilseed plants having reduced seedpod shatter). In some cases, mutagenesis (e.g., chemical mutagenesis) can be used to modify one or more genes encoding a polypeptide involved in seedpod shatter. As described herein, mutagenesis can be used to produce an oilseed plant having a loss-of-function modification in a SPT gene, a loss-of-function modification in a IND gene, a loss-of-function modification in one or more SHP genes, and/or a loss-of-function modification in a PID gene. In some cases, site-specific gene editing can be used to modify one or more genes encoding a polypeptide involved in seedpod shatter. As described herein, gene editing techniques (e.g., CRISPR-Cas systems) can be used to produce an oilseed plant having a loss-of-function modification in a ALC gene and/or a loss-of-function modification in an ADPG1 gene. One or more modifications to a gene encoding a polypeptide involved in seedpod shatter in a plant can be effective to cause a reduced polypeptide expression and/or polypeptide function, thereby reducing seedpod shatter in the plant.

The oilseed plants described herein (e.g., oilseed plants having reduced seedpod shatter) can be used as a cover crop that can reduce nutrient leaching, can reduce soil erosion, and/or can provide nectar and pollen provisions for pollinating insects such as bees.

The oilseed plants described herein (e.g., oilseed plants having reduced seedpod shatter) also can produce an economically viable product (e.g., edible oil, edible meal, and/or oil that can be used for the production of biodiesel, jet fuel, and/or other bioproducts).

The oilseed plants described herein (e.g., oilseed plants having reduced seedpod shatter) can be derived from any appropriate species of oilseed plant. An oilseed plant can be a monocotyledonous oilseed plant. An oilseed plant can be a dicotyledonous oilseed plant. An oilseed plant can be a member of the family Brassicaceae (e.g., the mustard family). For example, an oilseed plant can be a member of the genus Brassica. Examples of oilseed plants include, without limitation, pennycress, rapeseed, soybean, sunflower, canola, flax, camelina, carinata, lepidium, and crambe plants. In some cases, a domesticated oilseed plant having reduced seedpod shatter as described herein can be a pennycress plant.

In some cases, oilseed plants provided herein can have reduced seedpod (e.g., seedpod) shatter. The term “reduced seedpod shatter” as used herein with respect to seedpods of an oilseed plant refers to the seedpod requiring a greater amount of force to break open than an amount of force needed to break open a wild type seedpod. It will be appreciated that comparable oilseed plants are used when determining whether or not a particular oilseed plant has reduced seedpod shatter. Reduced seedpod shatter can also be referred to as, for example, increased pod shatter resistance. For example, a seedpod of a wild type pennycress plant typically shatters under about 3 grams to about 20 grams of force (e.g., pulling force). In some cases, a pennycress plant having reduced seedpod shatter as described herein can have seedpods that require greater than about 10 (e.g., greater than about 20, greater than about 30, greater than about 40, greater than about 50, greater than about 60, greater than about 70, greater than about 80, greater than about 90, greater than about 100, greater than about 110, or greater than about 120) grams force (e.g., pulling force) to shatter. In some cases, an oilseed plant having reduced seedpod shatter as described herein can have seedpods that require about 20 grams to about 120 grams (e.g., about 20 grams to about 100 grams, about 20 grams to about 90 grams, about 20 grams to about 80 grams, about 20 grams to about 60 grams, about 20 grams to about 45 grams, about 25 grams to about 120 grams, about 30 grams to about 120 grams, about 40 grams to about 120 grams, about 50 grams to about 120 grams, about 70 grams to about 120 grams, about 100 grams to about 120 grams, about 25 grams to about 100 grams, about 30 grams to about 90 grams, about 40 grams to about 80 grams, or about 50 grams to about 70 grams) force (e.g., pulling force) to shatter. For example, a pennycress plant having reduced seedpod shatter as described herein can have seedpods that require about 20 grams to about 45 grams pulling force to shatter. For example, a pennycress plant having reduced seedpod shatter as described herein can have seedpods that require about 40 grams to about 120 grams pulling force to shatter. In some cases, an oilseed plant having reduced seedpod shatter as described herein can have seedpods that are resistant to shatter under about 10 grams to about 30 grams pulling force. For example, a pennycress plant having reduced seedpod shatter as described herein can have seedpods that are resistant to shatter under less than about 30 (e.g., less than about 25, less than about 20, less than about 18, less than about 15, less than about 12, less than about 10, or less than about 7) grams force (e.g., pulling force). The oilseed plants having reduced seedpod shatter as described herein can be identified by, for example, measuring shatter resistance (e.g., as described in the Examples).

The oilseed plants having reduced seedpod shatter as described herein can be from the A7-129, A7-236, E42, ME3, ME5, or ME6 line as described, for example, in the Examples, or can be progeny derived from those lines.

The oilseed plants having reduced seedpod shatter as described herein can include any appropriate type of modification(s) in one or more genes that encode polypeptides involved in seedpod shatter. For example, a modification can be a loss-of-function modification. As used herein, a loss-of-function modification can be any modification that is effective to reduce polypeptide expression or polypeptide function. In some cases, reduced polypeptide expression or reduced polypeptide function can be eliminated polypeptide expression or eliminated polypeptide function. Examples of genetic modifications include, without limitation, deletions, insertions, substitutions, translocations, inversions, and duplications.

The oilseed plants having reduced seedpod shatter as described herein can include one or more modification(s) in any appropriate gene that encodes any appropriate polypeptide involved in seedpod shatter. Gens that encode polypeptides involved in seedpod shatter include, without limitation, SPT, alcatraz (ALC), indehiscent (IND), replumless (RPL), shatterproof (SHP; e.g., SHP1 and SHP2), fruitfull (FUL), nac secondary wall thickening promoting factor1 and 3 (NST1 and NST3), pinoid (PID), and dz polygalacturonase 1 and 2 (ADPG1 and ADPG2). Polypeptides involved in seedpod shatter include, without limitation, SPT, ALC, IND, RPL, SHP (e.g., SHP1 and SHP2), FUL, NST1, NST3, PID, ADPG1, and ADPG2.

In some cases, oilseed plants having reduced seedpod shatter as described herein can include one or more modifications (e.g., one or more loss-of-function modifications) in a SPT gene (e.g., in a SPT coding sequence) such that the one or more modifications are effective to reduce SPT polypeptide expression and/or SPT polypeptide function. A representative wild type pennycress SPT gene (e.g., coding sequence) is as set forth in SEQ ID NO:1.

In some cases, a wild type pennycress SPT gene (e.g., coding sequence) can have a sequence that deviates from the sequence set in SEQ ID NO:1, sometimes referred to as a variant sequence, provided the variant sequence encodes a wild type pennycress SPT polypeptide. A representative wild type pennycress SPT polypeptide is as set forth in SEQ ID NO:2.
In some cases, a wild type pennycress SPT polypeptide can have a sequence that deviates from the polypeptide sequence set forth in SEQ ID NO:2, sometimes referred to as a variant sequence, provided the polypeptide maintains its wild type function. For example, a variant SPT polypeptide can have at least 80 (e.g., at least 85, at least 90, at least 95, at least 98, or at least 99) percent sequence identity to SEQ ID NO:2. For example, a variant SPT polypeptide can have one or more (e.g., 2, 3, 4, 5, 6, 7, 8, 9, or 10) amino acid modifications (e.g., substitutions) relative to SEQ ID NO:2.

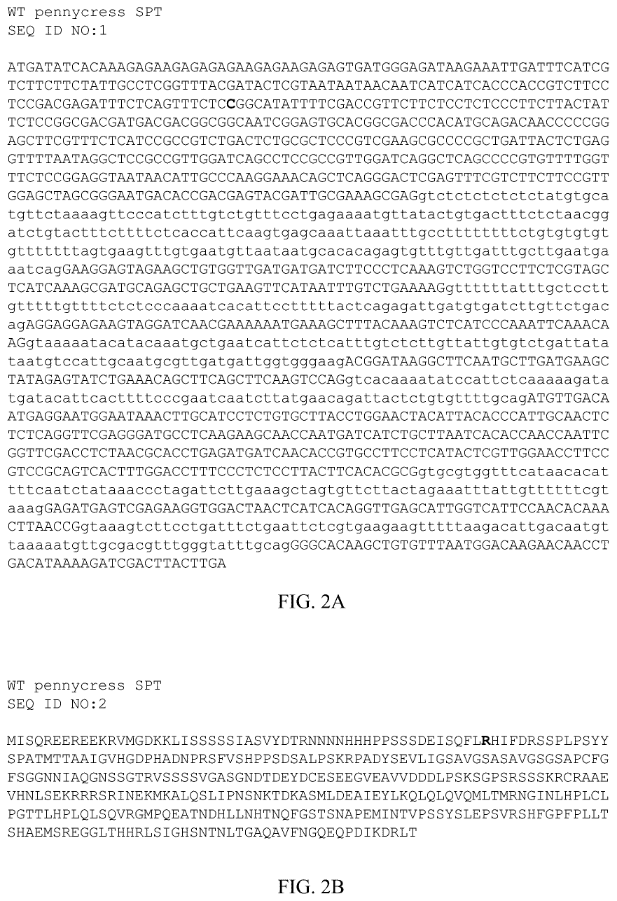

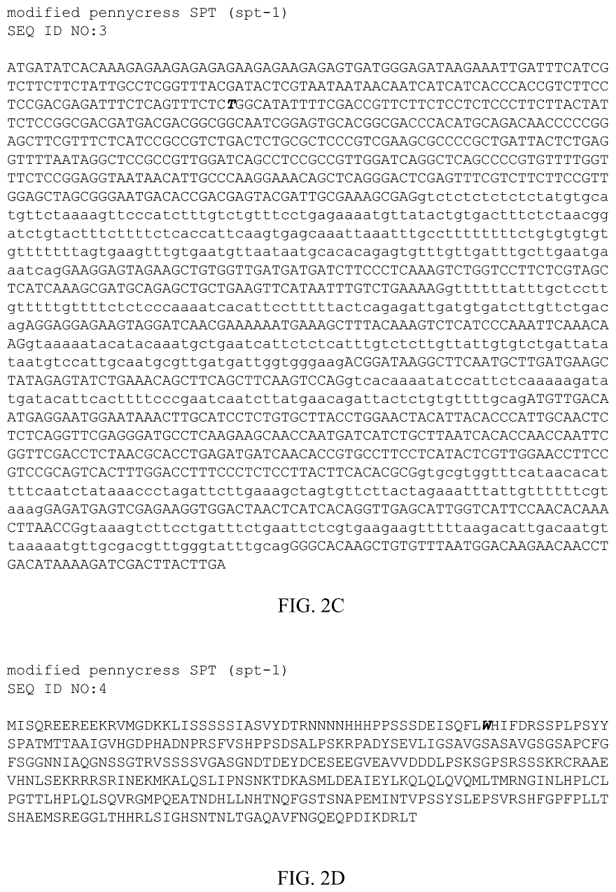

In some cases, oilseed plants described herein (e.g., oilseed plants having reduced seedpod shatter) can have one or more modifications in an SPT gene (e.g., coding sequence). For example, a pennycress plant having reduced seedpod shatter can include a substitution (e.g., a single base-pair substitution) relative to the wild type pennycress SPT gene (e.g., coding sequence). In some cases, a modified SPT gene can include a single base-pair substitution of the cytosine (C) at nucleotide residue 157 in a wild type pennycress SPT gene (e.g., SEQ ID NO:1). The C at nucleotide residue 157 can be substituted with any appropriate nucleotide (e.g., thymine (T), adenine (A), and guanine (G)). For example, a single base-pair substitution can be a C to T substitution at nucleotide residue 157 in a wild type pennycress SPT gene (see, e.g., FIG. 2). A representative modified pennycress SPT gene having a loss-of-function single base pair substitution is as set forth in SEQ ID NO:3. In some cases, oilseed plants having reduced seedpod shatter as described herein (e.g., oilseed plants having reduced seedpod shatter) can have one or more modifications in an SPT polypeptide (e.g., an SPT polypeptide encoded by an SPT gene having one or more modifications). For example, a modified pennycress SPT gene (e.g., coding sequence) having a loss-of-function single base pair substitution (e.g., SEQ ID NO:3) can encode a modified SPT polypeptide (e.g., a modified SPT polypeptide having reduced SPT polypeptide expression and/or reduced SPT polypeptide function). For example, a modified pennycress SPT gene having a single base-pair substitution (e.g., SEQ ID NO:3) can encode a modified SPT polypeptide. In some cases, a modified SPT polypeptide can include a substitution of the arginine (R) at amino acid residue 53 in a wild type pennycress SPT protein (e.g., SEQ ID NO:2). The R at residue 53 can be substituted with any appropriate amino acid (e.g., tryptophan (W)). For example, a modified SPT polypeptide can include a single W substituted for the R at amino acid residue 53 in a wild type pennycress SPT polypeptide (see, e.g., FIG. 2). A representative modified pennycress SPT polypeptide is as set forth in SEQ ID NO:4.

In some cases, oilseed plants having reduced seedpod shatter as described herein can include one or more modifications (e.g., one or more loss-of-function modifications) in a ALC gene (e.g., in a ALC coding sequence) such that the one or more modifications are effective to reduce ALC polypeptide expression and/or ALC polypeptide function. A representative wild type pennycress ALC gene (e.g., coding sequence) is as set forth in SEQ ID NO:5. In some cases, a wild type pennycress ALC gene (e.g., coding sequence) can have a sequence that deviates from the sequence set forth in SEQ ID NO:5, sometimes referred to as a variant sequence, provided the variant sequence encodes a wild type pennycress ALC polypeptide. A representative wild type pennycress ALC polypeptide is as set forth in SEQ ID NO:6. In some cases, a wild type pennycress ALC polypeptide can have a sequence that deviates from the polypeptide sequence set forth above (SEQ ID NO:6), sometimes referred to as a variant sequence, provided the polypeptide maintains its wild type function. For example, a ALC polypeptide can have at least 80 (e.g., at least 85, at least 90, at least 95, at least 98, or at least 99) percent sequence identity to SEQ ID NO:6. An ALC polypeptide can have one or more (e.g., 2, 3, 4, 5, 6, 7, 8, 9, or 10) amino acid modifications (e.g., substitutions) relative to SEQ ID NO:6.

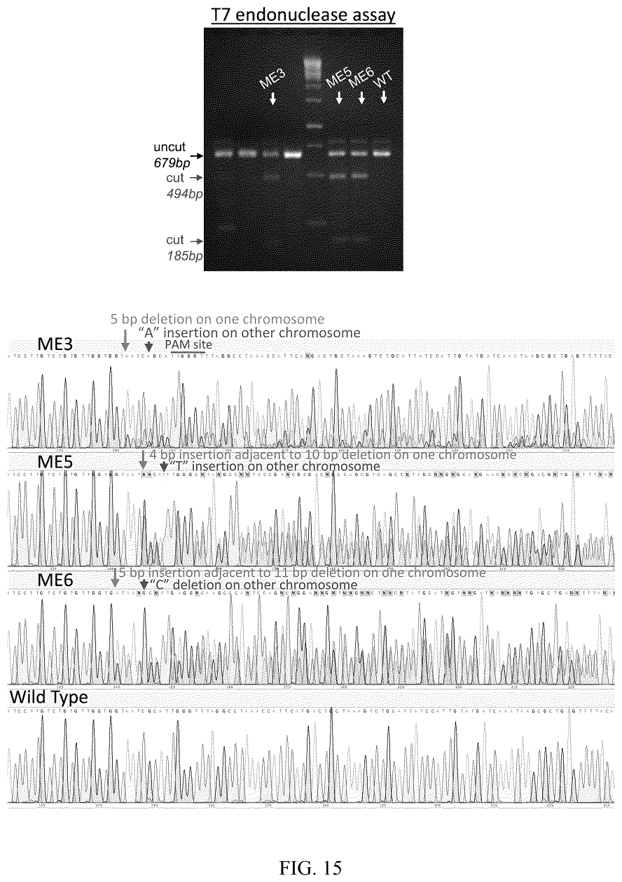

In some cases, oilseed plants described herein (e.g., oilseed plants having reduced seedpod shatter) can have one or more modifications in an ALC gene (e.g., coding sequence). For example, a pennycress plant having reduced seedpod shatter can include a deletion and/or an insertion (e.g., a combination of a deletion and an insertion in the location of the deletion) relative to the wild type pennycress ALC gene (e.g., coding sequence). The deletion can include any number of nucleotides (e.g., 1, 2, 3, 4, 5, 6, 7, 8, 9, 10, 11, 12, 13, 14 or 15 nucleotides). For example, a deletion can be a 5 base pair deletion or a 10 base pair deletion (see, e.g., FIG. 15). The insertion can include any appropriate number of nucleotides (e.g., 1, 2, 3, 4, 5, 6, 7, 8, or 9 nucleotides). For example, an insertion can be a 4 base pair insertion or a 5 base pair insertion (see, e.g., FIG. 15). The insertion can include any appropriate nucleotides (e.g., T, A, G, or C) in any appropriate sequence. For example, a 4 base pair insertion can have the sequence TCTC. In some cases, an oilseed plant having reduced seedpod shatter as described herein can include a 10 base pair deletion in a wild type pennycress ALC gene (e.g., SEQ ID NO:5) and a 4 base pair insertion in the location of the deletion. For example, an oilseed plant having reduced seedpod shatter as described herein can include a 10 base pair deletion of residues 827 to 836 in a wild type pennycress ALC gene, and a 4 base pair TCTC insertion following nucleotide residue 827 in a wild type pennycress ALC gene. A representative modified pennycress ALC gene (e.g., coding sequence) having a loss-of-function 4 base pair insertion in the location of a 10 base pair deletion is as set forth in SEQ ID NO:7. In some cases, oilseed plants having reduced seedpod shatter as described herein (e.g., oilseed plants having reduced seedpod shatter) can have one or more modifications in an ALC polypeptide (e.g., an ALC polypeptide encoded by an ALC gene having one or more modifications). For example, a modified pennycress ALC gene (e.g., coding sequence) having a loss-of-function 10 base pair deletion and a 4 base pair insertion in the location of the deletion (e.g., SEQ ID NO:7) can encode a modified ALC polypeptide (e.g., a modified ALC polypeptide having reduced ALC polypeptide expression and/or reduced ALC polypeptide function). In some cases, a modified pennycress ALC gene having a loss-of-function 10 base pair deletion and a 4 base pair insertion in the location of the deletion can encode a modified ALC polypeptide having a deletion of four amino acids, and an insertion of two different amino acids (e.g., SEQ ID NO:8). The deletion can include any number of amino acids (e.g., 1, 2, 3, 4, 5, 6, 7, 8, or 9 amino acids). For example, a deletion can be a 4 amino acid deletion. Any sequence of amino acids can be deleted. For example, the amino acids at residues 160-163 in a wild type pennycress ALC protein (e.g., SEQ ID NO:6) can be deleted. The insertion can include any appropriate number of amino acids (e.g., 1, 2, 3, or 4 amino acids). For example, an insertion can be a 2 amino acid insertion. The insertion can include any appropriate amino acids (e.g., isoleucine (I), serine (S), or W) in any appropriate sequence. For example, a 2 amino acid insertion can have the sequence IS. In some cases, a modified ALC polypeptide can include a deletion of the amino acid sequence asparagine-proline-methionine-arginine (NPMR) at residues 160-163 in a wild type pennycress ALC protein (e.g., SEQ ID NO:6), and an insertion of the amino acid sequence isoleucine-serine (IS) at residues 160-161 in the wild type pennycress ALC protein. A representative modified pennycress ALC polypeptide is as set forth in SEQ ID NO:8.

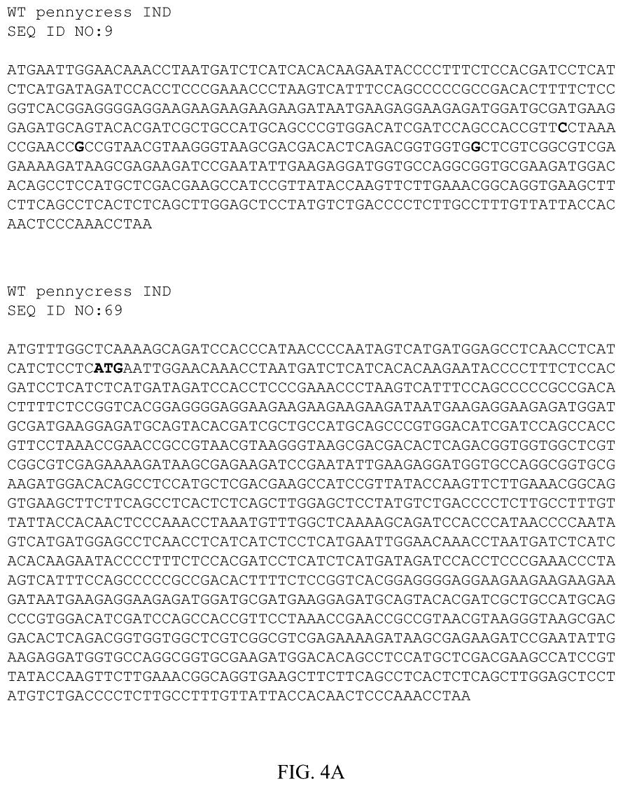

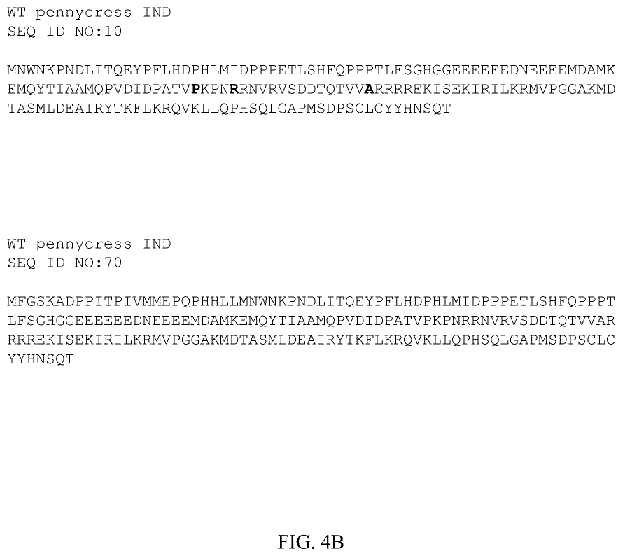

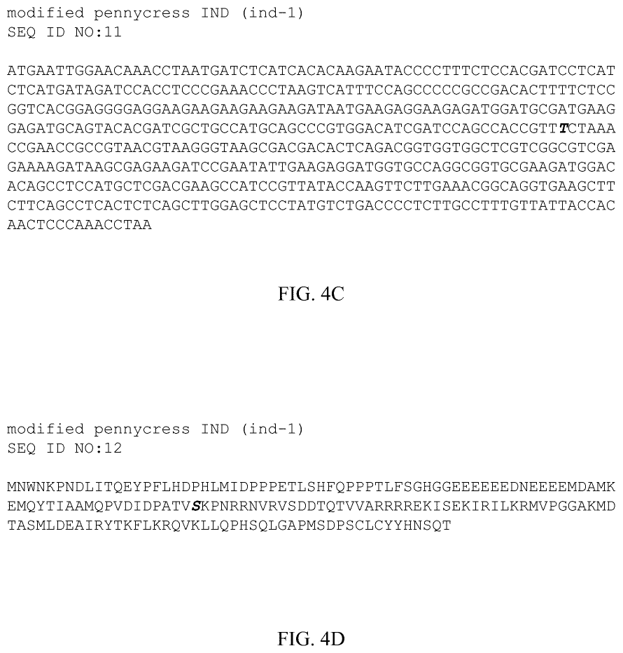

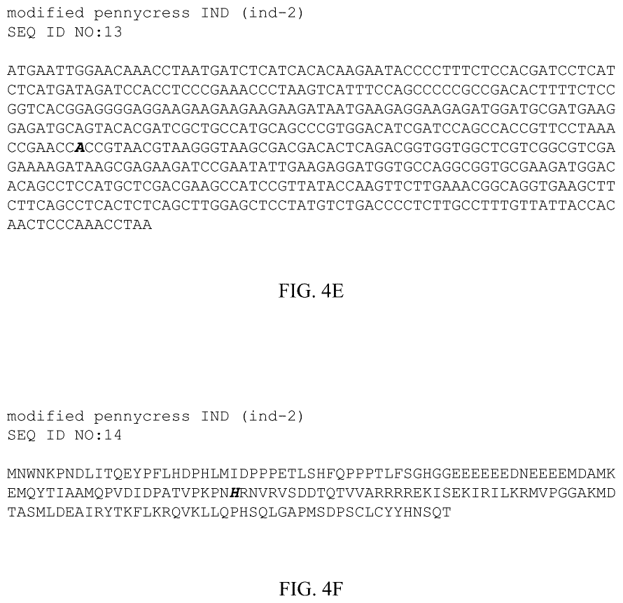

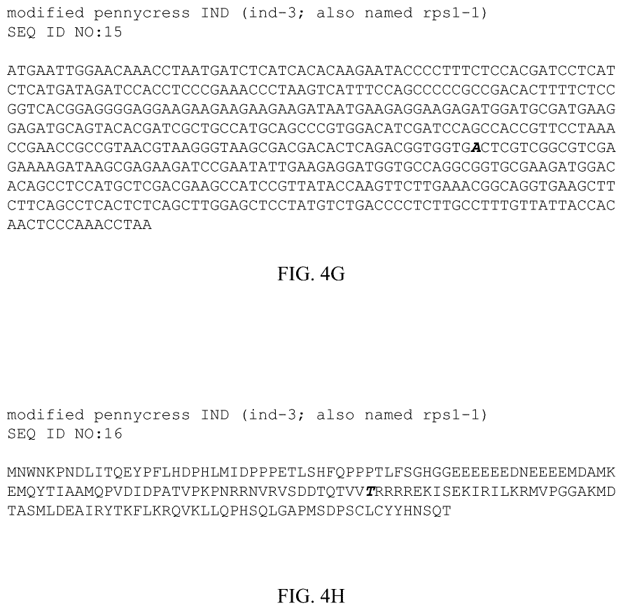

In some cases, oilseed plants having reduced seedpod shatter as described herein can include one or more modifications (e.g., one or more loss-of-function modifications) in a IND gene (e.g., in a IND coding sequence) such that the one or more modifications are effective to reduce IND polypeptide expression and/or IND polypeptide function. Representative wild type pennycress IND genes (e.g., coding sequences) are as set forth in SEQ ID NO:9 and SEQ ID NO:69. In some cases, a wild type pennycress IND gene (e.g., coding sequence) can have a sequence that deviates from the sequence set in SEQ ID NO:9 or SEQ ID NO:69, sometimes referred to as a variant sequence, provided the variant sequence encodes a wild type pennycress IND polypeptide. Representative wild type pennycress IND polypeptides are as set forth in SEQ ID NO:10 and SEQ ID NO:70. In some cases, a wild type pennycress IND polypeptide can have a sequence that deviates from the polypeptide sequence set forth in SEQ ID NO:10 or SEQ ID NO:70, sometimes referred to as a variant sequence, provided the polypeptide maintains its wild type function. For example, a variant IND polypeptide can have at least 80 (e.g., at least 85, at least 90, at least 95, at least 98, or at least 99) percent sequence identity to SEQ ID NO:10 or SEQ ID NO:70. For example, a variant IND polypeptide can have one or more (e.g., 2, 3, 4, 5, 6, 7, 8, 9, or 10) amino acid modifications (e.g., substitutions) relative to SEQ ID NO:10 or SEQ ID NO:70. In some cases, oilseed plants described herein (e.g., oilseed plants having reduced seedpod shatter) can have one or more modifications in an IND gene (e.g., coding sequence). For example, a pennycress plant having reduced seedpod shatter can include a substitution (e.g., a single base-pair substitution) relative to the wild type pennycress IND gene (e.g., coding sequence). In some cases, a modified IND gene can include a single base-pair substitution of the C at nucleotide residue 247 in a wild type pennycress IND gene (e.g., SEQ ID NO:9). The C at nucleotide residue 247 can be substituted with any appropriate nucleotide (e.g., T, A, and G). For example, a single base-pair substitution can be a C to T substitution at nucleotide residue 247 in a wild type pennycress IND gene (see, e.g., FIG. 4). A representative modified pennycress IND gene having a loss-of-function single base pair substitution is as set forth in SEQ ID NO:11. In some cases, oilseed plants having reduced seedpod shatter as described herein (e.g., oilseed plants having reduced seedpod shatter) can have one or more modifications in an IND polypeptide (e.g., an IND polypeptide encoded by an IND gene having one or more modifications). For example, a modified pennycress IND gene (e.g., coding sequence) having a loss-of-function single base pair substitution (e.g., SEQ ID NO:11) can encode a modified IND polypeptide (e.g., a modified IND polypeptide having reduced IND polypeptide expression and/or reduced IND polypeptide function). For example, a modified pennycress IND gene having a single base-pair substitution (e.g., SEQ ID NO:11) can encode a modified IND polypeptide. In some cases, a modified IND polypeptide can include a substitution of the proline (P) at amino acid residue 83 in a wild type pennycress IND protein (e.g., SEQ ID NO:10). The P at residue 83 can be substituted with any appropriate amino acid (e.g., serine (S)). For example, a modified IND polypeptide can include a single S substituted for the P at amino acid residue 83 in a wild type pennycress IND polypeptide (see, e.g., FIG. 4). A representative modified pennycress IND polypeptide is as set forth in SEQ ID NO:12. In some cases, a modified pennycress IND gene can have a modification as described relative to SEQ ID NO:9 in the corresponding residue in SEQ ID NO:69. In some cases, a modified pennycress IND polypeptide can have a modification as described relative to SEQ ID NO:10 in the corresponding residue in SEQ ID NO:70.

In some cases, a modified IND gene can include a single base-pair substitution of the G at nucleotide residue 260 in a wild type pennycress IND gene (e.g., SEQ ID NO:9). The G at nucleotide residue 260 can be substituted with any appropriate nucleotide (e.g., T, A, and C). For example, a single base-pair substitution can be a G to A substitution at nucleotide residue 260 in a wild type pennycress IND gene (see, e.g., FIG. 4). A representative modified pennycress IND gene having a loss-of-function single base pair substitution is as set forth in SEQ ID NO:13. In some cases, oilseed plants having reduced seedpod shatter as described herein (e.g., oilseed plants having reduced seedpod shatter) can have one or more modifications in an IND polypeptide (e.g., an IND polypeptide encoded by an IND gene having one or more modifications). For example, a modified pennycress IND gene (e.g., coding sequence) having a loss-of-function single base pair substitution (e.g., SEQ ID NO:13) can encode a modified IND polypeptide (e.g., a modified IND polypeptide having reduced IND polypeptide expression and/or reduced IND polypeptide function). For example, a modified pennycress IND gene having a single base-pair substitution (e.g., SEQ ID NO:13) can encode a modified IND polypeptide. In some cases, a modified IND polypeptide can include a substitution of the arginine (R) at amino acid residue 87 in a wild type pennycress IND protein (e.g., SEQ ID NO:10). The F at residue 87 can be substituted with any appropriate amino acid (e.g., histidine (H)). For example, a modified IND polypeptide can include a single H substituted for the R at amino acid residue 87 in a wild type pennycress IND polypeptide (see, e.g., FIG. 4). A representative modified pennycress IND polypeptide is as set forth in SEQ ID NO:14. In some cases, a modified pennycress IND gene can have a modification as described relative to SEQ ID NO:9 in the corresponding residue in SEQ ID NO:69. In some cases, a modified pennycress IND polypeptide can have a modification as described relative to SEQ ID NO:10 in the corresponding residue in SEQ ID NO:70.

In some cases, a modified IND gene can include a single base-pair substitution of the A at nucleotide residue 301 in a wild type pennycress IND gene (e.g., SEQ ID NO:9). The A at nucleotide residue 301 can be substituted with any appropriate nucleotide (e.g., T, G, and C). For example, a single base-pair substitution can be a G to A substitution at nucleotide residue 301 in a wild type pennycress IND gene (see, e.g., FIG. 4). A representative modified pennycress IND gene having a loss-of-function single base pair substitution is as set forth in SEQ ID NO:15. In some cases, oilseed plants having reduced seedpod shatter as described herein (e.g., oilseed plants having reduced seedpod shatter) can have one or more modifications in an IND polypeptide (e.g., an IND polypeptide encoded by an IND gene having one or more modifications). For example, a modified pennycress IND gene (e.g., coding sequence) having a loss-of-function single base pair substitution (e.g., SEQ ID NO:15) can encode a modified IND polypeptide (e.g., a modified IND polypeptide having reduced IND polypeptide expression and/or reduced IND polypeptide function). For example, a modified pennycress IND gene having a single base-pair substitution (e.g., SEQ ID NO:15) can encode a modified IND polypeptide. In some cases, a modified IND polypeptide can include a substitution of the alanine (A) at amino acid residue 101 in a wild type pennycress IND protein (e.g., SEQ ID NO:10). The A at residue 101 can be substituted with any appropriate amino acid (e.g., threonine (T)). For example, a modified IND polypeptide can include a single T substituted for the A at amino acid residue 101 in a wild type pennycress IND polypeptide (see, e.g., FIG. 4). A representative modified pennycress IND polypeptide is as set forth in SEQ ID NO:16. In some cases, a modified pennycress IND gene can have a modification as described relative to SEQ ID NO:9 in the corresponding residue in SEQ ID NO:69. In some cases, a modified pennycress IND polypeptide can have a modification as described relative to SEQ ID NO:10 in the corresponding residue in SEQ ID NO:70.

In some cases, sequences of modified IND genes and modified IND polypeptides can be as described elsewhere (see, e.g., Girin et al., 2010, Plant J., 63:329-338).

In some cases, sequences of modified RPL genes and modified RPL polypeptides can be as described elsewhere (see, e.g., Roeder et al., 2003, Curr. Biol., 13:1630-1635).

In some cases, oilseed plants having reduced seedpod shatter as described herein can include one or more modifications (e.g., one or more loss-of-function modifications) in a SHP1 gene (e.g., in a SHP1 coding sequence) such that the one or more modifications are effective to reduce SHP1 polypeptide expression and/or SHP1 polypeptide function. A representative wild type pennycress SHP1 gene (e.g., coding sequence) is as set forth in SEQ ID NO:17. In some cases, a wild type pennycress SHP1 gene (e.g., coding sequence) can have a sequence that deviates from the sequence set in SEQ ID NO:17, sometimes referred to as a variant sequence, provided the variant sequence encodes a wild type pennycress SHP1 polypeptide. A representative wild type pennycress SHP1 polypeptide is as set forth in SEQ ID NO:18. In some cases, a wild type pennycress SHP1 polypeptide can have a sequence that deviates from the polypeptide sequence set forth in SEQ ID NO:18, sometimes referred to as a variant sequence, provided the polypeptide maintains its wild type function. For example, a variant SHP1 polypeptide can have at least 80 (e.g., at least 85, at least 90, at least 95, at least 98, or at least 99) percent sequence identity to SEQ ID NO:18. For example, a variant SHP1 polypeptide can have one or more (e.g., 2, 3, 4, 5, 6, 7, 8, 9, or 10) amino acid modifications (e.g., substitutions) relative to SEQ ID NO:18.

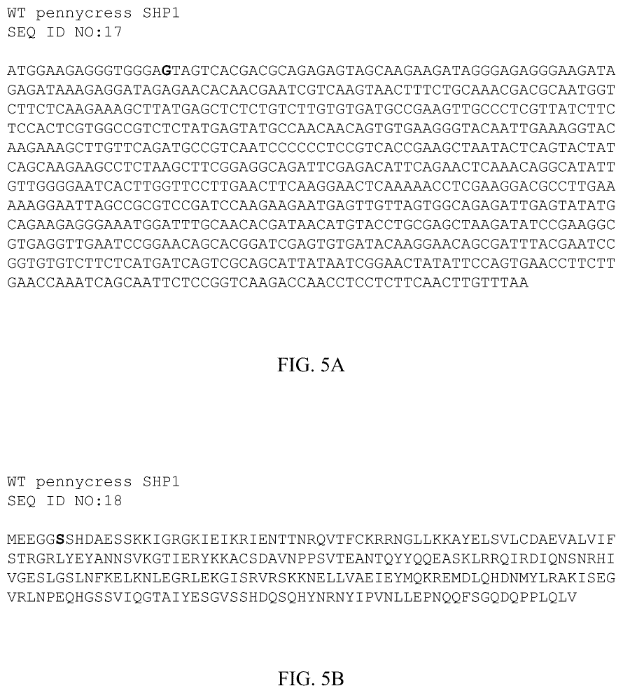

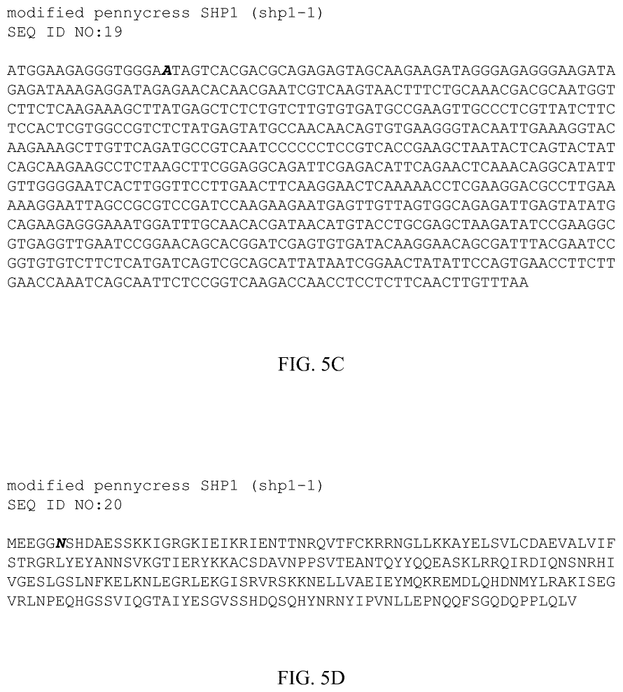

In some cases, oilseed plants described herein (e.g., oilseed plants having reduced seedpod shatter) can have one or more modifications in an SHP1 gene (e.g., coding sequence). For example, a pennycress plant having reduced seedpod shatter can include a substitution (e.g., a single base-pair substitution) relative to the wild type pennycress SHP1 gene (e.g., coding sequence). In some cases, a modified SHP1 gene can include a single base-pair substitution of the G at nucleotide residue 17 in a wild type pennycress SHP1 gene (e.g., SEQ ID NO:17). The G at nucleotide residue 17 can be substituted with any appropriate nucleotide (e.g., T, A, and C). For example, a single base-pair substitution can be a G to A substitution at nucleotide residue 17 in a wild type pennycress SHP1 gene (see, e.g., FIG. 5). A representative modified pennycress SHP1 gene having a loss-of-function single base pair substitution is as set forth in SEQ ID NO:19. In some cases, oilseed plants having reduced seedpod shatter as described herein (e.g., oilseed plants having reduced seedpod shatter) can have one or more modifications in an SHP1 polypeptide (e.g., an SHP1 polypeptide encoded by an SHP1 gene having one or more modifications). For example, a modified pennycress SHP1 gene (e.g., coding sequence) having a loss-of-function single base pair substitution (e.g., SEQ ID NO:19) can encode a modified SHP1 polypeptide (e.g., a modified SHP1 polypeptide having reduced SHP1 polypeptide expression and/or reduced SHP1 polypeptide function). For example, a modified pennycress SHP1 gene having a single base-pair substitution (e.g., SEQ ID NO:19) can encode a modified SHP1 polypeptide. In some cases, a modified SHP1 polypeptide can include a substitution of the serine (S) at amino acid residue 6 in a wild type pennycress SHP1 protein (e.g., SEQ ID NO:18). The S at residue 6 can be substituted with any appropriate amino acid (e.g., asparagine (N)). For example, a modified SHP1 polypeptide can include a single S substituted for the S at amino acid residue 6 in a wild type pennycress SHP1 polypeptide (see, e.g., FIG. 5). A representative modified pennycress SHP1 polypeptide is as set forth in SEQ ID NO:20. In some cases, sequences of modified SHP1 genes and modified SHP1 polypeptides can be as described elsewhere (see, e.g., Liljegren et al., 2000, Nature, 404:766-770).

In some cases, oilseed plants having reduced seedpod shatter as described herein can include one or more modifications (e.g., one or more loss-of-function modifications) in a SHP2 gene (e.g., in a SHP2 coding sequence) such that the one or more modifications are effective to reduce SHP2 polypeptide expression and/or SHP1 polypeptide function. A representative wild type pennycress SHP2 gene (e.g., coding sequence) is as set forth in SEQ ID NO:21. In some cases, a wild type pennycress SHP2 gene (e.g., coding sequence) can have a sequence that deviates from the sequence set in SEQ ID NO:21, sometimes referred to as a variant sequence, provided the variant sequence encodes a wild type pennycress SHP2 polypeptide. A representative wild type pennycress SHP2 polypeptide is as set forth in SEQ ID NO:22. In some cases, a wild type pennycress SHP2 polypeptide can have a sequence that deviates from the polypeptide sequence set forth in SEQ ID NO:22, sometimes referred to as a variant sequence, provided the polypeptide maintains its wild type function. For example, a variant SHP2 polypeptide can have at least 80 (e.g., at least 85, at least 90, at least 95, at least 98, or at least 99) percent sequence identity to SEQ ID NO:22. For example, a variant SHP2 polypeptide can have one or more (e.g., 2, 3, 4, 5, 6, 7, 8, 9, or 10) amino acid modifications (e.g., substitutions) relative to SEQ ID NO:22.

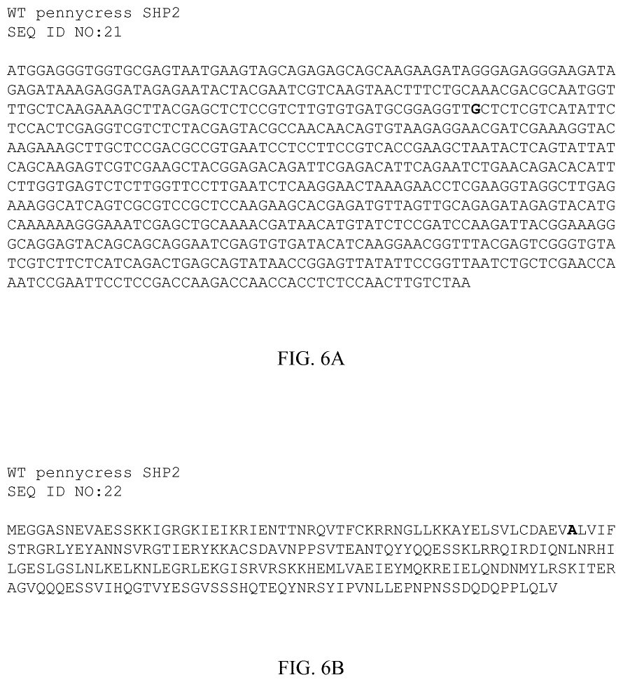

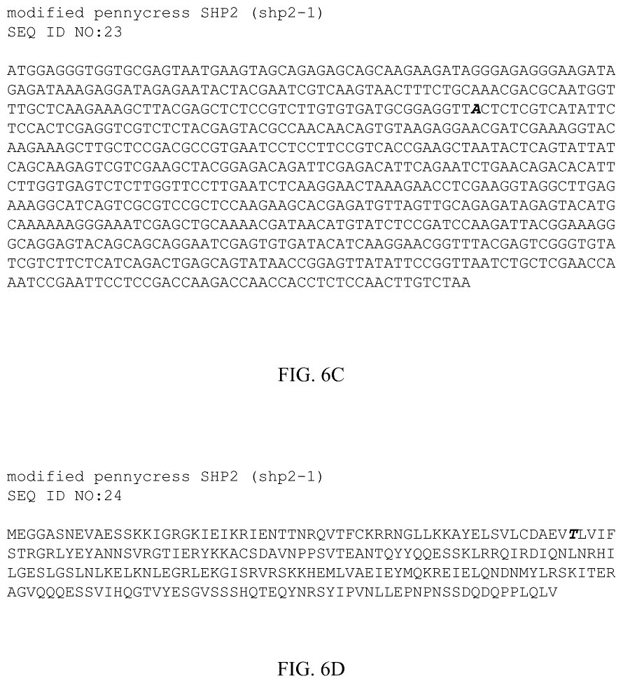

In some cases, oilseed plants described herein (e.g., oilseed plants having reduced seedpod shatter) can have one or more modifications in an SHP2 gene (e.g., coding sequence). For example, a pennycress plant having reduced seedpod shatter can include a substitution (e.g., a single base-pair substitution) relative to the wild type pennycress SHP2 gene (e.g., coding sequence). In some cases, a modified SHP2 gene can include a single base-pair substitution of the G at nucleotide residue 175 in a wild type pennycress SHP2 gene (e.g., SEQ ID NO:21). The G at nucleotide residue 175 can be substituted with any appropriate nucleotide (e.g., T, A, and C). For example, a single base-pair substitution can be a G to A substitution at nucleotide residue 175 in a wild type pennycress SHP2 gene (see, e.g., FIG. 6). A representative modified pennycress SHP2 gene having a loss-of-function single base pair substitution is as set forth in SEQ ID NO:23. In some cases, oilseed plants having reduced seedpod shatter as described herein (e.g., oilseed plants having reduced seedpod shatter) can have one or more modifications in an SHP2 polypeptide (e.g., an SHP2 polypeptide encoded by an SHP2 gene having one or more modifications). For example, a modified pennycress SHP2 gene (e.g., coding sequence) having a loss-of-function single base pair substitution (e.g., SEQ ID NO:23) can encode a modified SHP2 polypeptide (e.g., a modified SHP2 polypeptide having reduced SHP2 polypeptide expression and/or reduced SHP2 polypeptide function). For example, a modified pennycress SHP2 gene having a single base-pair substitution (e.g., SEQ ID NO:23) can encode a modified SHP2 polypeptide. In some cases, a modified SHP2 polypeptide can include a substitution of the alanine (A) at amino acid residue 59 in a wild type pennycress SHP2 protein (e.g., SEQ ID NO:22). The A at residue 59 can be substituted with any appropriate amino acid (e.g., threonine (T)). For example, a modified SHP2 polypeptide can include a single T substituted for the A at amino acid residue 59 in a wild type pennycress SHP2 polypeptide (see, e.g., FIG. 6). A representative modified pennycress SHP2 polypeptide is as set forth in SEQ ID NO:24. In some cases, sequences of modified SHP2 genes and modified SHP2 polypeptides can be as described elsewhere (see, e.g., Liljegren et al., 2000, Nature, 404:766-770).

In some cases, sequences of modified FUL genes and modified FUL polypeptides can be as described elsewhere (see, e.g., Ferrándiz et al., 2000, Science, 289:436-438).

In some cases, oilseed plants having reduced seedpod shatter as described herein can include one or more modifications (e.g., one or more loss-of-function modifications) in a PID gene (e.g., in a PID coding sequence) such that the one or more modifications are effective to reduce PID polypeptide expression and/or PID polypeptide function. A representative wild type pennycress PID gene (e.g., coding sequence) is as set forth in SEQ ID NO:25. In some cases, a wild type pennycress PID gene (e.g., coding sequence) can have a sequence that deviates from the sequence set in SEQ ID NO:25, sometimes referred to as a variant sequence, provided the variant sequence encodes a wild type pennycress PID polypeptide. A representative wild type pennycress PID polypeptide is as set forth in SEQ ID NO:26. In some cases, a wild type pennycress PID polypeptide can have a sequence that deviates from the polypeptide sequence set forth in SEQ ID NO:26, sometimes referred to as a variant sequence, provided the polypeptide maintains its wild type function. For example, a variant PID polypeptide can have at least 80 (e.g., at least 85, at least 90, at least 95, at least 98, or at least 99) percent sequence identity to SEQ ID NO:26. For example, a variant PID polypeptide can have one or more (e.g., 2, 3, 4, 5, 6, 7, 8, 9, or 10) amino acid modifications (e.g., substitutions) relative to SEQ ID NO:26.

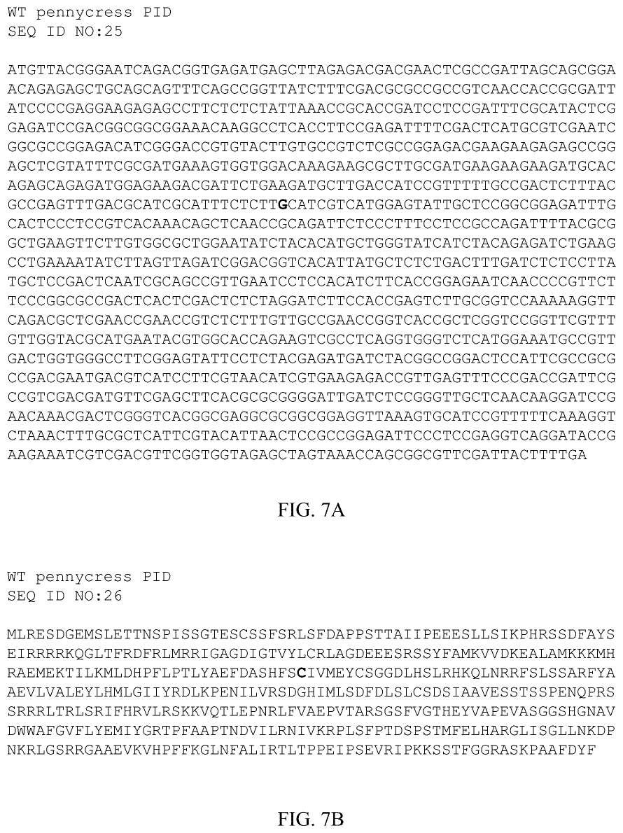

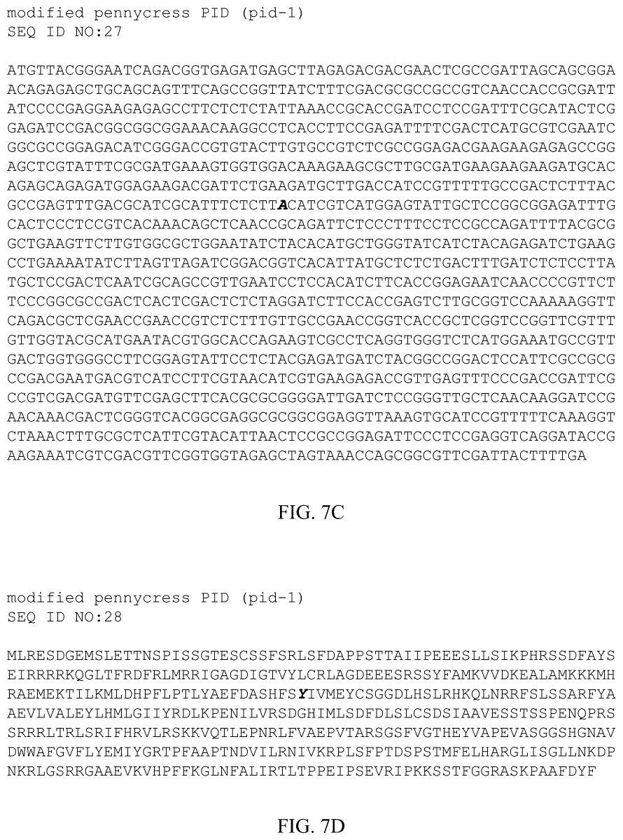

In some cases, oilseed plants described herein (e.g., oilseed plants having reduced seedpod shatter) can have one or more modifications in a PID gene (e.g., coding sequence). For example, a pennycress plant having reduced seedpod shatter can include a substitution (e.g., a single base-pair substitution) relative to the wild type pennycress PID gene (e.g., coding sequence). In some cases, a modified PID gene can include a single base-pair substitution of the G at nucleotide residue 470 in a wild type pennycress PID gene (e.g., SEQ ID NO:25). The G at nucleotide residue 470 can be substituted with any appropriate nucleotide (e.g., T, A, and C). For example, a single base-pair substitution can be a G to A substitution at nucleotide residue 470 in a wild type pennycress PID gene (see, e.g., FIG. 7). A representative modified pennycress PID gene having a loss-of-function single base pair substitution is as set forth in SEQ ID NO:27. In some cases, oilseed plants having reduced seedpod shatter as described herein (e.g., oilseed plants having reduced seedpod shatter) can have one or more modifications in a PID polypeptide (e.g., a PID polypeptide encoded by a PID gene having one or more modifications). For example, a modified pennycress PID gene (e.g., coding sequence) having a loss-of-function single base pair substitution (e.g., SEQ ID NO:27) can encode a modified PID polypeptide (e.g., a modified PID polypeptide having reduced PID polypeptide expression and/or reduced PID polypeptide function). For example, a modified pennycress PID gene having a single base-pair substitution (e.g., SEQ ID NO:27) can encode a modified PID polypeptide. In some cases, a modified PID polypeptide can include a substitution of the cysteine (C) at amino acid residue 157 in a wild type pennycress PID protein (e.g., SEQ ID NO:26). The C at residue 157 can be substituted with any appropriate amino acid (e.g., tyrosine (Y)). For example, a modified PID polypeptide can include a single Y substituted for the C at amino acid residue 157 in a wild type pennycress PID polypeptide (see, e.g., FIG. 7). A representative modified pennycress PID polypeptide is as set forth in SEQ ID NO:28.

In some cases, oilseed plants having reduced seedpod shatter as described herein can include one or more modifications (e.g., one or more loss-of-function modifications) in a ADPG1 gene (e.g., in a ADPG1 coding sequence) such that the one or more modifications are effective to reduce ADPG1 polypeptide expression and/or ADPG1 polypeptide function. A representative wild type pennycress ADPG1 gene (e.g., coding sequence) is as set forth in SEQ ID NO:29. In some cases, a wild type pennycress ADPG1 gene (e.g., coding sequence) can have a sequence that deviates from the sequence set in SEQ ID NO:29, sometimes referred to as a variant sequence, provided the variant sequence encodes a wild type pennycress ADPG1 polypeptide. A representative wild type pennycress ADPG1 polypeptide is as set forth in SEQ ID NO:30. In some cases, a wild type pennycress ADPG1 polypeptide can have a sequence that deviates from the polypeptide sequence set forth in SEQ ID NO:30, sometimes referred to as a variant sequence, provided the polypeptide maintains its wild type function. For example, a variant ADPG1 polypeptide can have at least 80 (e.g., at least 85, at least 90, at least 95, at least 98, or at least 99) percent sequence identity to SEQ ID NO:30. For example, a variant ADPG1 polypeptide can have one or more (e.g., 2, 3, 4, 5, 6, 7, 8, 9, or 10) amino acid modifications (e.g., substitutions) relative to SEQ ID NO:30.

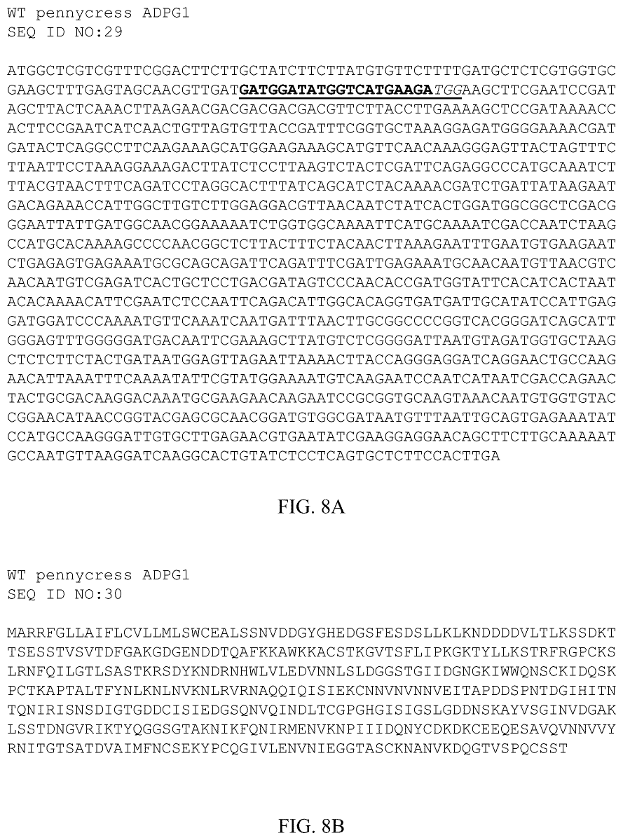

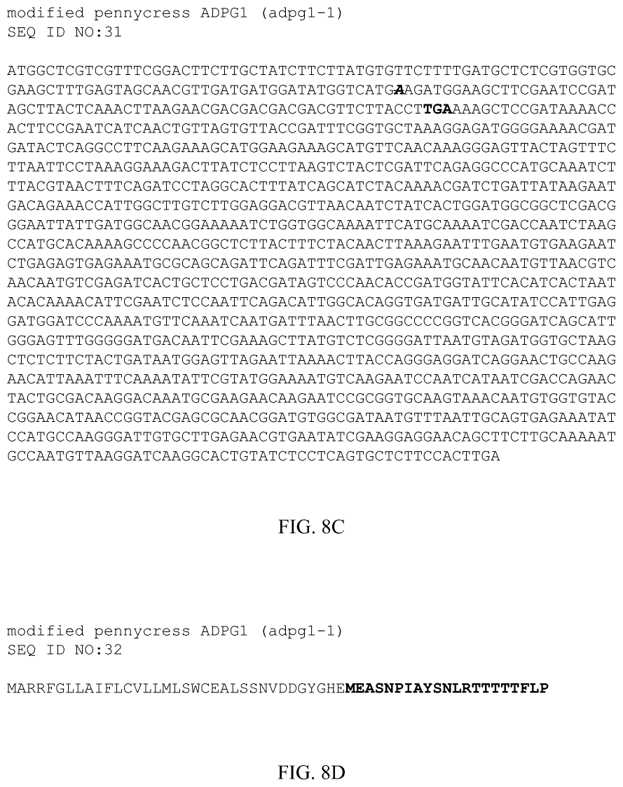

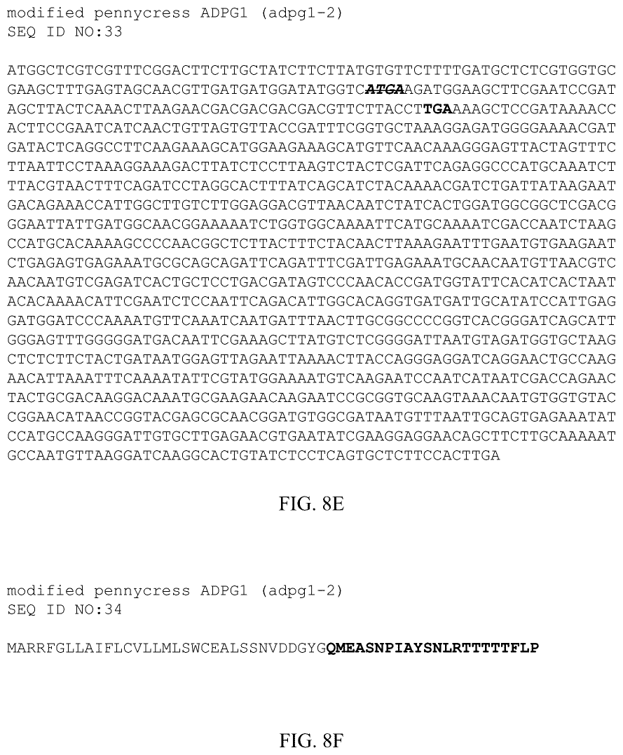

In some cases, oilseed plants having reduced seedpod shatter as described herein (e.g., oilseed plants having reduced seedpod shatter) can have one or more modifications in an ADPG1 gene (e.g., coding sequence). For example, a pennycress plant having reduced seedpod shatter can include a deletion relative to the wild type pennycress ALC gene (e.g., coding sequence). The deletion can include any number of nucleotides (e.g., 1, 2, 3, 4, 5, 6, 7, 8, 9, 10, 11, 12, 13, 14 or 15 nucleotides). For example, a deletion can be a single base pair deletion or a 4 base pair deletion (see, e.g., FIG. 8). In some cases, an oilseed plant having reduced seedpod shatter as described herein can include a single base pair deletion in a wild type pennycress ADPG1 gene (e.g., SEQ ID NO:29). For example, an oilseed plant having reduced seedpod shatter as described herein can include a single base pair deletion of residue 104 in a wild type pennycress ADPG1 gene. A representative modified pennycress ALC gene (e.g., coding sequence) having a loss-of-function single base pair deletion is as set forth in SEQ ID NO:31. In some cases, oilseed plants having reduced seedpod shatter as described herein (e.g., oilseed plants having reduced seedpod shatter) can have one or more modifications in an ADPG1 polypeptide (e.g., an ADPG1 polypeptide encoded by an ADPG1 gene having one or more modifications). For example, a modified pennycress ADPG1 gene (e.g., coding sequence) having a loss-of-function single base pair deletion (e.g., SEQ ID NO:31) can encode a modified ADPG1 polypeptide (e.g., a modified ADPG1 polypeptide having reduced ADPG1 polypeptide expression and/or reduced ADPG1 polypeptide function). In some cases, a modified pennycress ADPG1 gene having a loss-of-function single base pair deletion can encode a modified ADPG1 polypeptide that is truncated relative to a wild type ADPG1 polypeptide (e.g., SEQ ID NO:30). In some cases, a single base pair deletion can result in a frameshift such that a modified ADPG1 polypeptide can include an aberrant amino acid sequence (e.g., an aberrant C-terminal amino acid sequence). A representative modified pennycress ALC polypeptide is as set forth in SEQ ID NO:32.

In some cases, an oilseed plant having reduced seedpod shatter as described herein can include a 4 base pair deletion in a wild type pennycress ADPG1 gene (e.g., SEQ ID NO:29). For example, an oilseed plant having reduced seedpod shatter as described herein can include a 4 base pair deletion of residues 101 to 104 in a wild type pennycress ADPG1 gene. A representative modified pennycress ALC gene (e.g., coding sequence) having a loss-of-function single base pair deletion is as set forth in SEQ ID NO:33. In some cases, oilseed plants having reduced seedpod shatter as described herein (e.g., oilseed plants having reduced seedpod shatter) can have one or more modifications in an ADPG1 polypeptide (e.g., an ADPG1 polypeptide encoded by an ADPG1 gene having one or more modifications). For example, a modified pennycress ADPG1 gene (e.g., coding sequence) having a loss-of-function 4 base pair deletion (e.g., SEQ ID NO:33) can encode a modified ADPG1 polypeptide (e.g., a modified ADPG1 polypeptide having reduced ADPG1 polypeptide expression and/or reduced ADPG1 polypeptide function). In some cases, a modified pennycress ADPG1 gene having a loss-of-function 4 base pair deletion can encode a modified ADPG1 polypeptide that is truncated relative to a wild type ADPG1 polypeptide (e.g., SEQ ID NO:30). In some cases, a 4 base pair deletion can result in a frameshift such that a modified ADPG1 polypeptide can include an aberrant amino acid sequence (e.g., an aberrant C-terminal amino acid sequence). A representative modified pennycress ALC polypeptide is as set forth in SEQ ID NO:34.

Any appropriate method can be used to introduce one or more modifications into a gene encoding a polypeptide involved in seedpod shatter to produce oilseed plants described herein (e.g., oilseed plants having reduced seedpod shatter).

In some cases, mutagenesis (e.g., chemical mutagenesis) can be used to produce oilseed plants having reduced seedpod shatter. For example, mutagenesis can be used to modify one or more genes encoding a polypeptide involved in seedpod shatter. Mutagenesis can be performed using any appropriate mutagen. A mutagen can be a chemical mutagen. Examples of mutagens that can be used to produce oilseed plants having reduced seedpod shatter include, without limitation, ethyl methane sulphonate (EMS), 1-methyl-1-nitrosourea, 1-ethyl-1-nitrosourea, fast neutrons (FN), gamma rays, x-rays, ultraviolet light, T-DNAs, RNA interference (RNAi), and micro RNAs. For example, EMS can be used to induce G to A substitutions and/or C to T substitutions in a nucleotide sequence. In cases where mutagenesis is used to produce oilseed plants having one or more modifications in a gene encoding a polypeptide involved in seedpod shatter, one or more genes encoding a polypeptide involved in seedpod shatter and/or one or more polypeptides involved in seedpod shatter can be sequenced to determine whether or not a modification described herein (e.g., a modification effective to result in reduced seedpod shatter) is present.

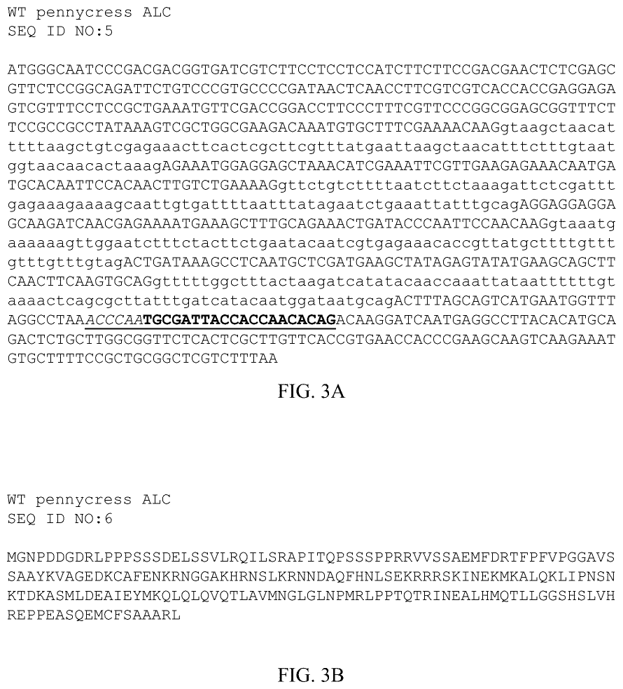

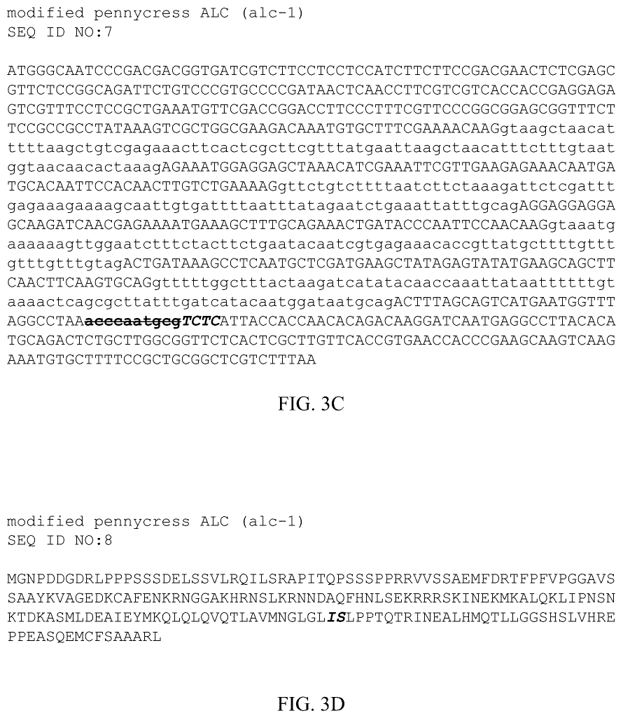

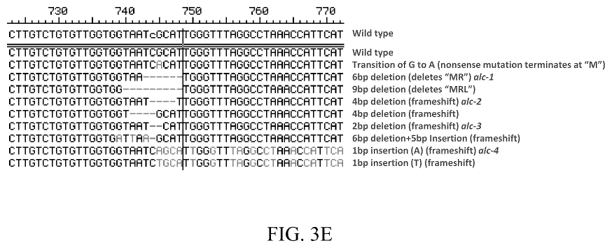

In some cases, genome editing can be used to produce oilseed plants having reduced seedpod shatter. For example, genome editing can be used to modify one or more genes encoding a polypeptide involved in seedpod shatter. Genome editing, or genome editing with engineered nucleases (GEEN) can insert, replace, or remove DNA from a genome using one or more site-specific nucleases (SSN) and, in some cases, a repair template (RT). Nucleases can be targeted to a specific position in the genome, where their action can introduce a particular modification to the endogenous sequences. For example, a SSN can introduce a targeted double-strand break (DSB) in the genome, such that cellular DSB repair mechanisms incorporate a RT into the genome in a configuration that produces heritable glyphosate resistance in the cell, in a plant regenerated from the cell, and in any progeny of the regenerated plant. Nucleases useful for genome editing include, without limitation, CRISPR-associated nucleases (e.g., Cas9, SpCas9, SaCas9, and Cpf1), zinc finger nucleases (ZFNs), transcription activator-like effector nucleases (TALE nucleases), and homing endonucleases (HE; also referred to as meganucleases). For example, a CRISPR-Cas9 system can be used to introduce one or more loss-of-function modifications described herein into a gene (e.g., coding sequence) encoding a polypeptide involved in seedpod shatter. For example, a CRISPR-Cas9 vector can include at least one guide sequence specific to a wild type pennycress ALC sequence (see, e.g., SEQ ID NO:5, FIG. 3, and Example 3) and/or at least one guide sequence specific to a pennycress ADPG1 sequence (see, e.g., SEQ ID NO:29, FIG. 8, and Example 4) upstream of a PAM. A Cas enzyme will bind to and cleave within the gene only if the target site is followed by a PAM sequence. For example, the canonical PAM is the sequence 5′-NGG-3′, where N is any nucleotide followed by two guanine (G) nucleotides. The Cas9 component of a CRISPR-Cas9 system designed to introduce one or more loss-of-function modifications described herein can be any appropriate Cas9. In some cases, the Cas9 of a CRISPR-Cas9 system described herein can be a Staphylococcus aureus Cas9 (SaCas9). One example of a SaCas9 is described in, for example, Steinert et al., 2015 Plant J. 84:1295-305.

The genome editing reagents described herein can be introduced into an oilseed plant by any appropriate method. In some cases, nucleic acids encoding the genome editing reagents can be introduced into a plant cell using Agrobacterium or Ensifer mediated transformation, particle bombardment, liposome delivery, nanoparticle delivery, electroporation, polyethylene glycol (PEG) transformation, or any other method suitable for introducing a nucleic acid into a plant cell. In some cases, the SSN or other expressed gene editing reagents can be delivered as RNAs or as proteins to a plant cell and the RT, if one is used, can be delivered as DNA.

The invention will be further described in the following examples, which do not limit the scope of the invention described in the claims.

### EXAMPLES

### Example 1: Domesticated Pennycress Plants

To identify domestication genes in pennycress plants, pennycress seeds were mutagenized with several different mutagens, including ethyl methanesulfonate (EMS), fast neutrons (FN) and gamma rays (γ rays). Treatment of dry plant seeds with mutagens results in the generation of distinct sets of mutations in every cell in the seed. The fate of many of these cells can be followed when a mutation in one of these cells results in a visible phenotype creating a marked plant sector. For example, mutations that induce the formation of either yellow or pale sectors have been used to generate embryonic cellular fate maps in Arabidopsis. These fate maps make predictions about the contribution of cells in the embryonic meristem to development of subsequent plant structures. The largest of such sectors can be used to estimate the number of cells (known as the genetically effective cells) that give rise to the gametes in the treated dry M1 seeds. In such sectors, both the pollen and egg cells share common progenitor cells. After meiosis in commonly derived anthers and ovules, one quarter of the seeds will be homozygous for any mutations carried in common progenitor cells. Sectors similar to those seen in mutagenized Arabidopsis populations were observed in the EMS mutagenized M1 pennycress plants. The largest sectors observed in pennycress encompassed approximately a third of the floral meristem, which is consistent with the presence of three genetically effective cells in the dry seed. Thus, a pool of seeds collected from 10 M1 plants would represent approximately 30 different mutagenic events.

Pennycress plants exhibiting domestication enabling traits such as reduced seedpod shatter were analyzed and loss of function mutations in domestication genes were identified.

### Materials and Methods

### Mutagenesis (EMS, FN, and Gamma Ray).

Seeds treated with 0.2% EMS showed somatic chlorophyll sectoring in the leaves (comparable to what is seen with EMS mutagenized Arabidopsis) and all of the treated plants set seeds. Approximately 40,000 seeds were treated with EMS and planted in the field. The seeds germinated and a stand of pennycress was established. Approximately 1-2% of the seedlings showed somatic chlorophyll sectoring in the leaves. In addition to EMS, batches of seeds (40,000 per batch) were exposed to different levels of fast neutrons and gamma rays. Seeds were treated with gamma rays with levels ranging from 5 to 45 kilorads (kR) and were treated with fast neutrons (FN) with levels from 8 to 30 gray (Gy). All treated seeds germinated at similar levels and some somatic sectors were noted in seedlings treated with 30 Gy FN. These seeds were considered to be the progenitors of the M1 generation of plants.

### Growth of Plants

Winter annual pennycress mutagenized M1 seeds were planted into small plots during the summer. In addition, M1 seeds from an EMS treated spring line were grown in growth chambers. M2 seeds were collected from mutagenized M1 plants and were either grown in the field or in growth chambers. The resulting M2 plants were screened for interesting morphological traits such as altered growth, early flowering and senescence, shatter resistance, and reduced stature. In addition, M3 seeds were collected from approximately 15,000 randomly selected individual M2 plants.

### Impact of Shatter in the Field

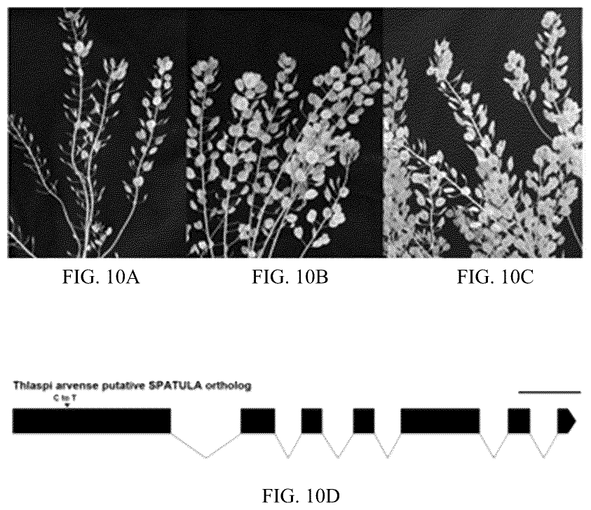

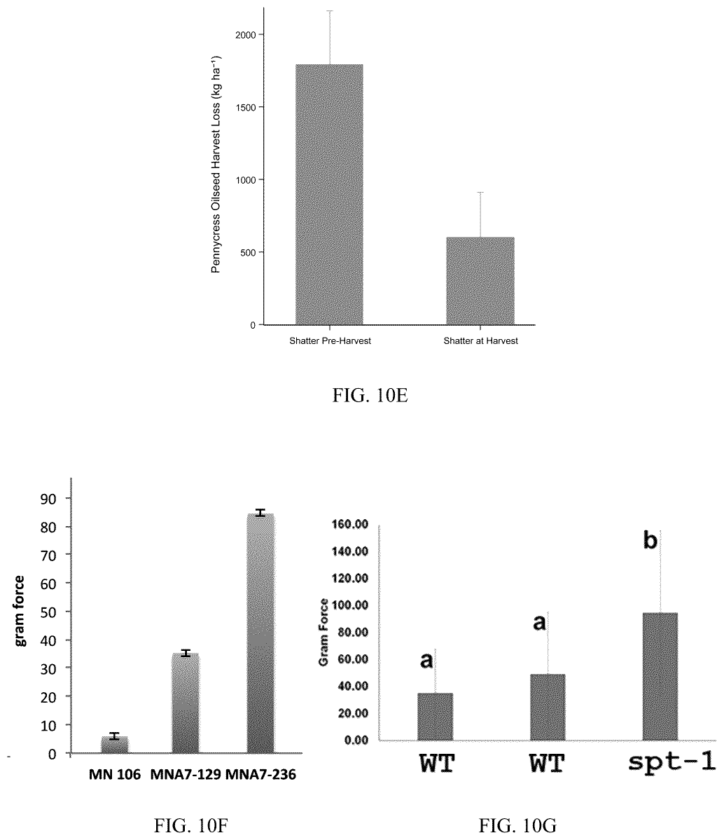

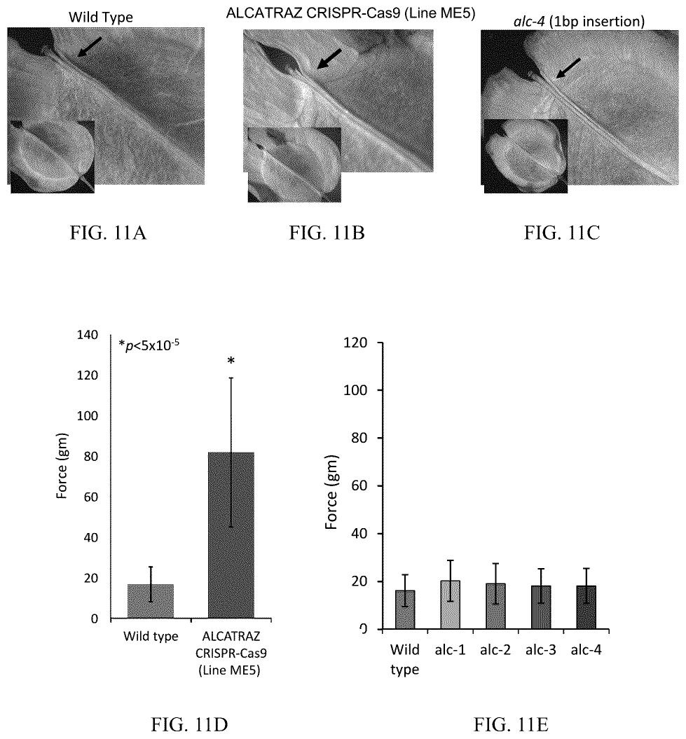

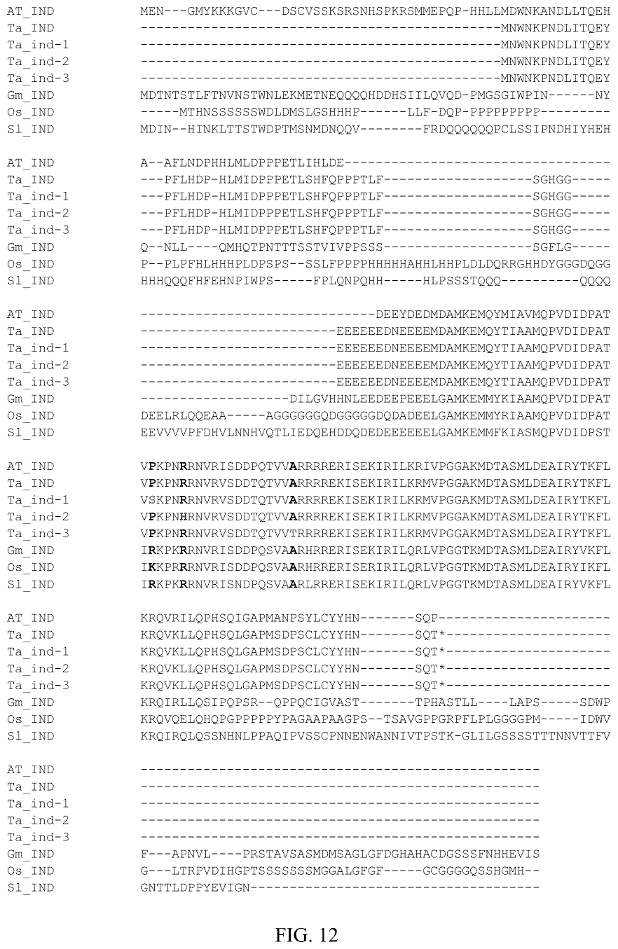

Images were taken of field grown wild type and mutant plants at a time past normal harvest date (FIGS. 2C-D). To measure pre harvest losses due to environmental conditions, plastic containing of known area were place under the canopy of wild type pennycress plants two weeks prior to normal harvest date. Weight of seeds that dropped pre harvest into the container divided by the container area were converted to kg/ha as shown in FIG. 10E. New plastic container were placed in the field immediately prior to harvest. Seeds that dropped into the containing during the harvest were used to calculate loss during harvest as shown in FIG. 10E.

### Measuring Shatter Resistance

Shatter resistance was quantitatively measured using a gram force meter attached to an alligator clip. One lateral side of a pod was clipped and the other side was manually pulled until the pod split apart at the septum. The maximum force recorded by the meter was consider the force needed to break open the pod.

### Sequencing

PCR primers were designed to amplify the candidate pennycress genes (Table 1) and the products were subject to Sanger sequencing.

TABLE 1
PCR primers.

| | forward | SEQ | reverse | SEQ |
|---|---|---|---|---|
| | primer | ID | primer | ID |
| primer | sequence | NO: | sequence | NO: |
| SPT | GGGCAATGTTATT | 51 | GGCTCTATGACAG | 52 |
| | ACCTCCG | | ACCAATC | |

### Results

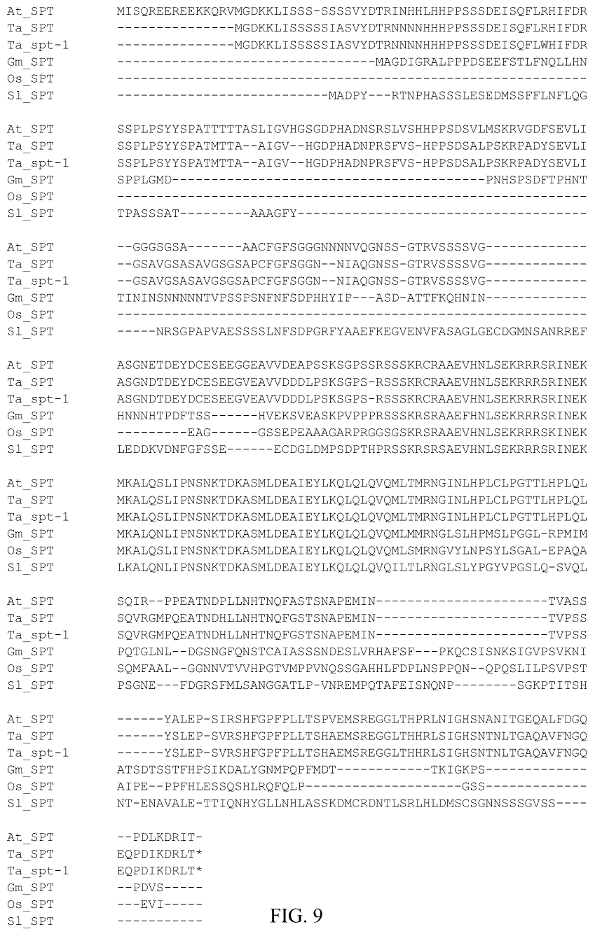

Pod shatter can drastically reduce yield, either from natural pod breakage in the field or due to losses during harvest (FIG. 10E). Several M2 lines showing reduced seedpod shatter were identified. The reanalysis of M3 progeny from two of these lines, MN A7-129 and MN A7-236, is shown in FIGS. 10F and 10G. The mutant pods required statistically significant greater force than wild type for breakage. Furthermore, these mutants exhibited reduced shattering under field conditions relative to wild type (FIGS. 10A-10C). WGS revealed the presence of a mutation in a putative Arabidopsis ortholog of SPATULA (SPT) in MN A7-129 (FIG. 9 and FIG. 10D). The mutation results in the substitution of tryptophan for a highly conserved arginine. Importantly in Arabidopsis, spt mutants exhibit increased pod shatter resistance.

These results demonstrate that a domesticated pennycress plant with reduced seedpod shatter can be designed by modifying the SPT gene.

### Example 2: Generation and Characterization of Indehiscent (Ind; Also Referred to as Reduced Pod Shatter1-1 (rps1-1)) Line E42

### Materials and Methods

### Solutions:

A) 0.2 M sodium phosphate monobasic (NaH₂PO₄*H₂O) 6.9 g/250 mL
B) 0.2 M sodium phosphate dibasic (NaH₂PO₄ anhydrous) 7.1 g/250 mL
For 50 mL of 0.1 M sodium phosphate buffer at pH 7:
   9.75 mL A
   15.25 mL B
   25.0 mL dH₂O
0.2% EMS in buffer:
   20 mL 0.1M Sodium Phosphate Buffer, pH 7
   40 μL EMS liquid (Sigma #M0880-5G)
0.1 M sodium thiosulfate at pH 7.3:
   12.4 g sodium thiosulfate in 500 mL

### Primary Seed Surface Sterilization

Wild-type pennycress (Thlaspi arvense) seeds (Spring 32 ecotype) were surface sterilized for 10 minutes in a 30% bleach, 0.05% SDS solution before being rinsed 3× with sterile water. Sterilized seeds were immediately subjected to EMS treatment.

### Ethyl Methane Sulfonate (EMS) Treatment of Pennycress Seeds

Sterilized pennycress seeds (41 g) were agitated in distilled water overnight. Four 250 mL, Erlenmeyer flasks with 10 g seed each, and 1 g in a separate small flask as a control, were agitated. The water was decanted.

25 mLs of 0.2% EMS in 0.1M sodium phosphate buffer (pH 7) was added. The control received only phosphate buffer with no EMS. The flasks were shaken in fume hood for 18 hours. The EMS solution was decanted off into an EMS waste bottle.

To rinse the seeds, 25 mLs of dH2O was added to each flask, and the flasks were shaken for 20 minutes. The rinse water was decanted into the EMS waste bottle.

To deactivate the EMS, seeds were washed for 20 minutes in 0.1M sodium thiosulfate (pH 7.3). The sodium thiosulfate solution was decanted into the EMS waste bottle.

The seeds were rinsed 4 times with dH2O for 15 minutes.

The seeds were suspended in 0.1% agarose, and germinated directly in autoclaved Reddiearth soil at a density of approximately 10 seeds per 4-inch pot.

### Plant Growth Conditions

EMS-treated pennycress seeds were germinated and grown in an environmental growth chamber at 21° C., 16:8 6400K fluorescent light/dark, 50% humidity. Approximately 14 days after planting, plants were thinned and transplanted to a density of 4 plants per 4-inch pot. These M₁-generation plants showed telltale chlorotic leaf sectors that are indicative of a successful mutagenesis.

After dry down, these M₁-generation plants were catalogued and harvested. The M₂-generation seeds were surface sterilized, planted and grown according to the protocols previously described.

### Results

### Identification and Characterization of RPS1-1 Mutant Line E42

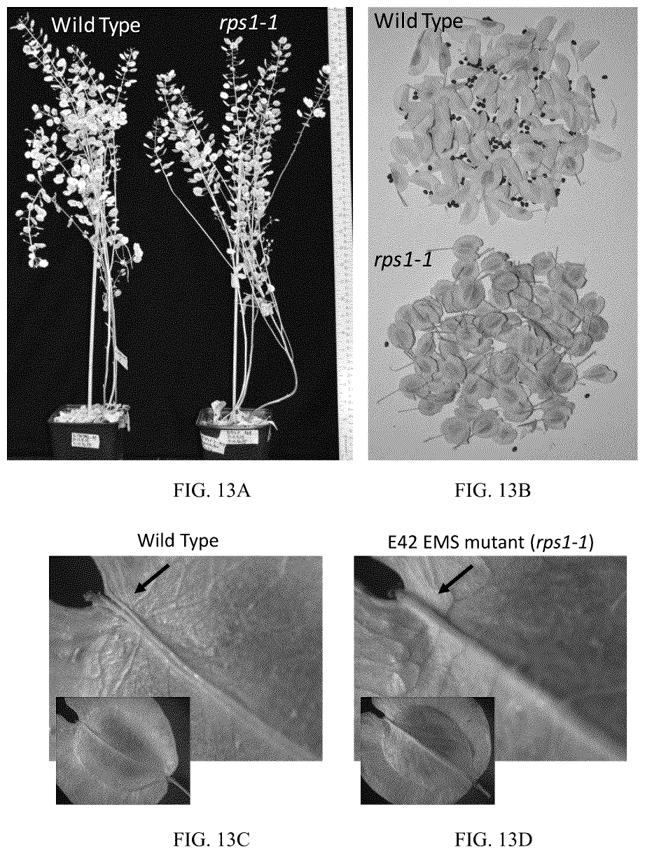

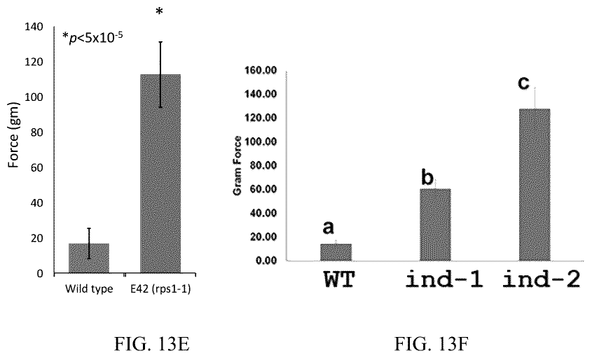

Putative reduced seedpod shatter mutants in the M₂-generation were identified as those requiring relatively more force to rupture the pods compared to wild type when harvesting with a closed hand. Seeds (M₃-generation) from putative M₂-generation mutants were planted and grown in potting soil-containing 4-inch pots in a growth chamber and the pod shatter phenotype re-assessed upon plant senescence. Siblings from only one line (line E42, herein named reduced pod shatter 1-1 or rps1-1; also herein named ind-3) reproduced an obvious reduced seedpod shatter phenotype (FIG. 13). That phenotype was also observed in over-wintered field-grown F₂-generation plants arising from the following crosses: rps1-1×wild-type Elizabeth; rps1-1×wild-type MN106.

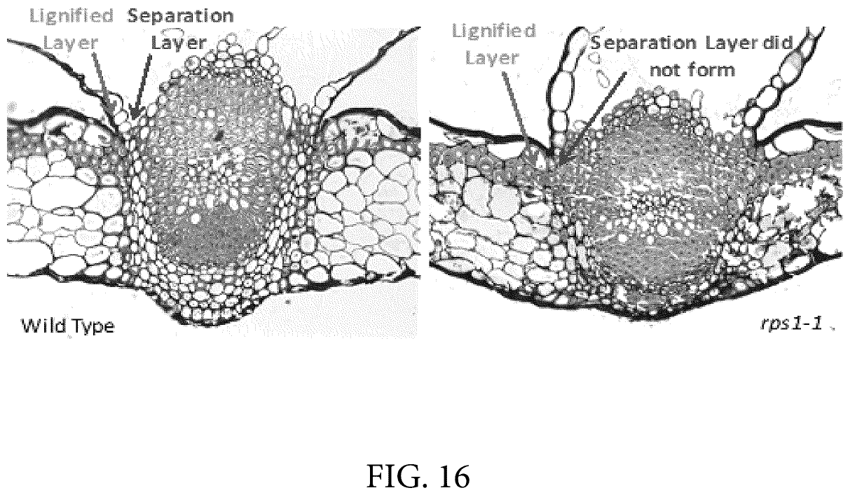

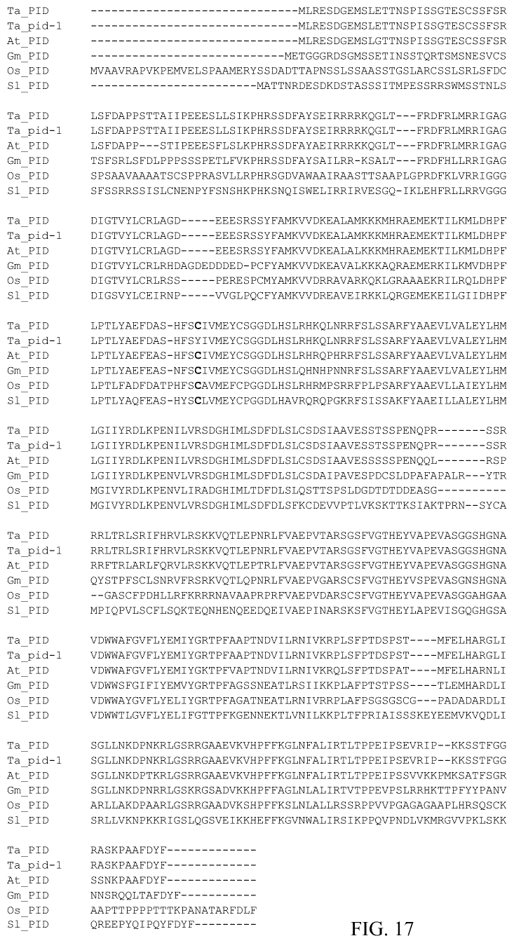

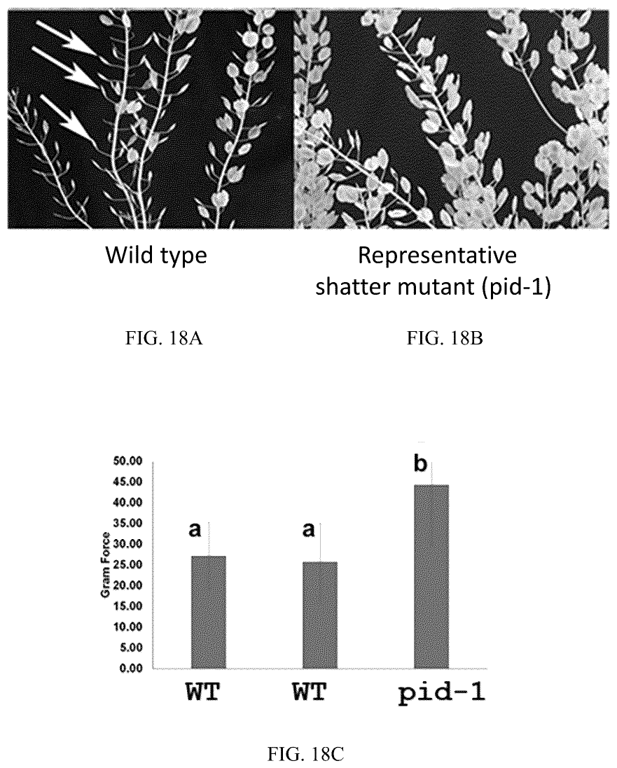

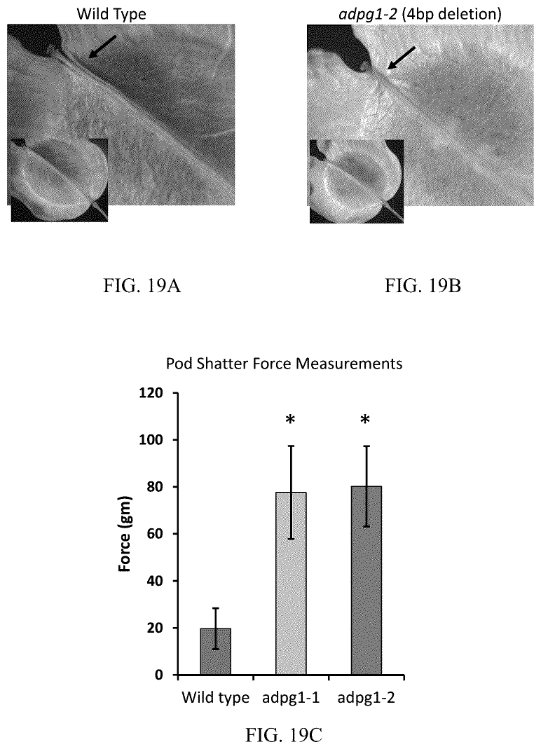

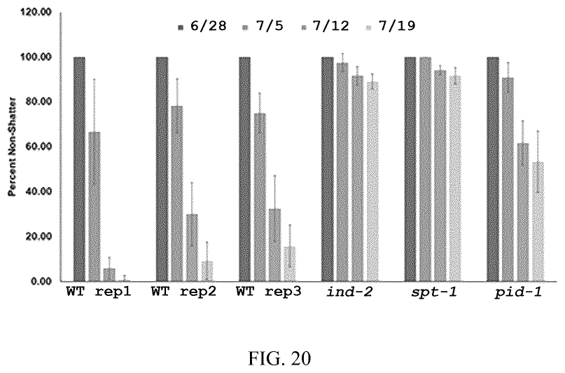

The growth of rps1-1 plants was indistinguishable from that of wild-type plants (FIG. 13). When pulling the senesced seedpods apart with fingers, rps1-1 pods did not break at the dehiscence zone like wild type, but instead tore within the valve. Microscopic examination of the seedpod dehiscence zone showed that wild type pods often-times were slightly breaking apart, which was a phenotype not observed in rps1-1 pods (FIG. 13). Microscopic examination of toluidine blue-stained sections of rps1-1 seedpods revealed the lignified layer and separation layer within the dehiscence zone were poorly formed or absent (FIG. 16).

Genetic crosses were also made between rps1-1 plants and wild-type Spring 32 plants. F₁-generation plants exhibited wild-type pod shatter whereas F₂-generation plants segregated 3:1 wild-type to reduced seedpod shatter (61 plants out of 217 showed the pod shatter phenotype=28.1%). These results confirmed the rps1-1 mutation to be recessive and segregated in a Mendelian fashion.

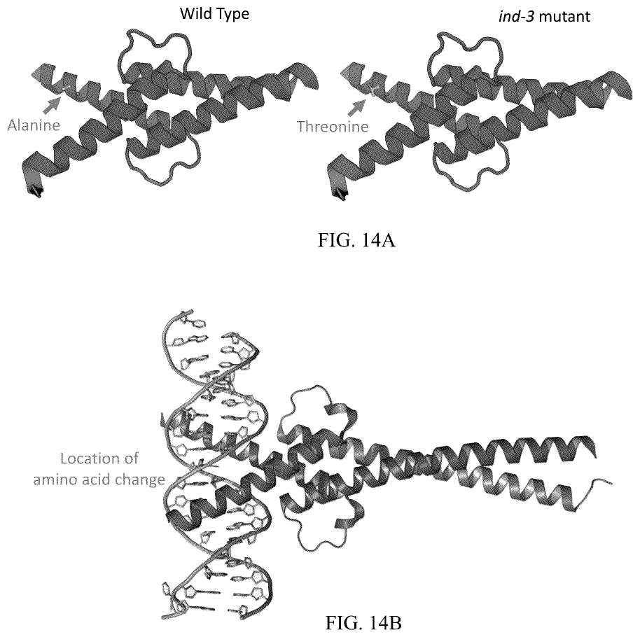

EMS mutagenesis typically introduces single-nucleotide transition mutations (e.g. G to A) into plant genomes. To identify the mutation in rps1-1 plants, DNA was extracted from rps1-1 and wild-type leaf tissue using either the CTAB protocol (below) or the Qiagen DNeasy Plant Mini Kit (product number 69106). PCR amplification of the INDEHISCENT (IND; also referred to as REDUCED SEEDPOD SHATTER (RPS)), SPATULA (SPT), ALCATRAZ (ALC), and REPLUMLESS (RPL) genes was performed and the resultant DNA sequenced. No mutations were initially identified in these genes. To resolve this, DNA from 42 F₂-generation rps1-1×wild-type Spring 32 plants was extracted using the Qiagen DNeasy Plant Mini Kit and pooled. The pooled DNA was sequenced using a HiSeq 2500 sequencer. Detailed sequence analyses confirmed that a G to A transition mutation in the IND gene coding sequence co-segregated with the reduced seedpod shatter phenotype. This transition mutation at nucleotide residue 301 as set forth in SEQ ID NO:15 results in an alanine (A) to threonine (T) amino acid change at residue 101 in the amino acid sequence set forth in SEQ ID NO:16. The amino acid change resides in the DNA binding domain of the IND polypeptide (FIG. 14A, B) and likely disrupts binding of the IND transcription factor to promoters of seedpod shatter-related genes it regulates.

### Example 3: Shatter Generation and Characterization of ALC Mutant Lines ME3, ME5, and ME6 by Direct Targeting of ALC with CRISPR-SaCas9

### Materials and Methods

### Construction of the Pennycress ALCATRAZ (ALC) Gene-Specific CRISPR-saCas9 Vector

The constructs and cloning procedures for generation of Thlaspi arvense (pennycress) ALC-specific CRISPR-SaCas9 constructs were as described elsewhere (see, e.g., Steinert et al., 2015, Plant J., 84:1295-305; and Fauser et al., 2014, Plant J., 79: 348-359).

The plant selectable marker (formerly NPT) was replaced with a hygromycin resistance (Hygromycin phosphotransferase (HPT)) gene in the pDe-SaCas9 binary vector.

Oligos were annealed to create a 20mer protospacer specific to the pennycress ALC sequence:

### PennyALC_CRISPR-SaCAS9_FWD:

(SEQ ID NO: 53)
5′ ATTGTGCGATTACCACCAACACAG 3′

### PennyALC_CRISPR-SaCAS9_REV:

(SEQ ID NO: 54)
5′ AAACCTGTGTTGGTGGTAATCGCA 3′

Vector Transformation into Agrobacterium

The pDe-SaCas9_Hyg vector containing the Staphylococcus aureus Cas9 (SaCas9) cassette with the pennycress ALC sequence-specific protospacer was transformed into Agrobacterium tumefaciens strain GV3101 using the freeze/thaw method described elsewhere (see, e.g., indiana.edu/˜pikweb/Protocols %20page.html)

The transformation product was plated on 1% agar Luria Broth (LB) plates with gentamycin (50 μg/ml) rifampicin (50 μg/ml) and spectinomycin (75 μg/ml). Single colonies were selected after two days of growth at 28° C.

### Plant Transformation—Pennycress Floral Dip

Day One:

5 mL of LB+5 uL with appropriate antibiotics (Rifampin (50), Spectinomycin (75), and/or Gentamycin (50)) were inoculated with Agrobacterium. The cultures were allowed to grow, with shaking, overnight at 28° C.

Day Two (Early Morning):

25 mL of Luria Broth+25 uL appropriate antibiotics (Rifampin (50), Spectinomycin (75), and/or Gentamycin (50)) were inoculated with the initial culture from day one. The cultures were allowed to grow, with shaking, overnight at 28° C.

Day Two (Late Afternoon):

250 mL of Luria Broth+250 uL appropriate antibiotic (Rifampin (50), Spectinomycin (75), and/or Gentamycin (50)) were inoculated with 25 mL culture. The cultures were allowed to grow, with shaking, overnight at 28° C.

Day Three:

When the culture had grown to an OD₆₀₀ of ˜1 (or looks thick and silky), the culture was decanted into large centrifuge tubes (all evenly weighted with analytical balance), and spun at 3,500 RPM at room temperature for 10 minutes to pellet cells. The supernatant was decanted off. The pelleted cells were resuspended in a solution of 5% sucrose and 0.02% Silwet L-77. The suspension was poured into clean beakers and placed in a vacuum chamber.

Newly flowering inflorescences of pennycress were fully submerged into the beakers, and subjected to a pressure of 30 PSI for 10 minutes.

After racemes of pennycress plants (Spring32 variety; these plants were 5 generations removed from seeds) were dipped, they were covered loosely with Saran wrap to maintain humidity and kept in the dark overnight before being uncovered and placed back in the environmental growth chamber.

### Screening Transgenic Plants and Growth Condition

Pennycress seeds were surface sterilized by first rinsing in 70% ethanol then incubating 10 minutes in a 30% bleach, 0.05% SDS solution before being rinsed two times with sterile water and plated on selective plates (0.8% agar/one half-strength Murashige and Skoog salts with hygromycin B selection at 40 U ml⁻¹. Plates were wrapped in parafilm and kept in an environmental growth chamber at 21° C., 16:8 day/night for 8 days until hygromycin selection was apparent.

Surviving hygromycin-resistant T1-generation seedlings were transplanted into autoclaved Reddiearth soil mix and grown in an environmental growth chamber set to 16 hour days/8 hour nights at 21° C. and 50% humidity. T2-generation seeds were planted, and ˜1.5 mg of leaf tissue from each T2-generation plant was harvested with a 3 mm hole punch, then processed using the Thermo Scientific™ Phire™ Plant Direct PCR Kit (Catalog #F130WH) as per manufacturer's instructions. PCR (20 μl volume) was performed on samples from six plants whose pods putatively required more hand force than wild type to shatter upon plant senescence. For the PCR, the following two primers were used, which amplified a 679 base pair fragment containing the protospacer location where the CRISPR-SaCas9 guide RNA and endonuclease had been targeted to bind and cut the genomic DNA:

ALCPCRF2:
(SEQ ID NO: 55)
AGGAGCTAAACATCGAAATTCGTTGAAGAG
ALCPCRR2:
(SEQ ID NO: 56)
TGTCGCAGATACTAGAGGAACATCACATCA

10 μl of the PCR product was digested with T7 Endonuclease I (Fisher Scientific catalog #M0302L) as per manufacturer's instructions then electrophoresed in a 1% agarose gel (FIG. 15). T7 endonuclease cleaves DNA that contains CRISPR-Cas9-induced DNA mismatches, in this case producing ˜185 base pair fragment and a ˜494 base pair fragment. The PCR products from all six plants were sequenced, and the three that gave positive results from the T7 endonuclease assay (lines ME3, ME5, and ME6) were found to have mutations in each chromosome pair in the locations expected for CRISPR-Cas9-induced mutations (e.g. at the protospacer adjacent to the NNGGGT PAM site where the guide was targeted to bind. Sequence chromatograms and descriptions of the mutations can be found in FIG. 15.

### Example 4: Shatter Generation and Characterization of ADPG1 Mutant Lines by Direct Targeting of ADPG1 with CRISPR-SaCas9

### Materials and Methods

The protocols used to generate ADPG1-specific CRISPR-SpCas9 constructs and to identify adpg1 mutations and mutant lines were the same as described in Example 4 above, except instead of using an SaCas9 vector, a modified SpCas9 vector as described in Fauser et al., 2014, Plant J., 79: 348-359 was used. As with the pDe-SaCas9 binary vector, we replaced the plant selectable marker (formerly NPT) in the pDe-SpCas9 binary vector with a hygromycin resistance (Hygromycin phosphotransferase (HPT)) gene. The ADPG1-targeting gRNA protospacer sequence that was used in this vector (highlighted in bold and underlined in SEQ ID NO:29), which is adjacent to the NGG PAM site (underlined and italicized in SEQ ID NO:29), is as follows:

### PennyADPG1_CRISPR-SpCAS9_FWD:

(SEQ ID NO: 57)
5′ GATGGATATGGTCATGAAGA 3′

### PennyADPG1_CRISPR-SpCAS9_REV:

(SEQ ID NO: 58)
5′ TCTTCATGACCATATCCATC 3′

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-24. (canceled)

25. A pennycress plant comprising a modification in an indehiscent (IND) gene, wherein an unmodified IND gene encodes a polypeptide having at least 95% sequence identity to SEQ ID NO:9, wherein the plant comprising the modified IND gene exhibits reduced seedpod shatter compared to a corresponding plant lacking the modification.

26. The plant of claim 25, wherein the modification comprises a substitution.

27. The plant of claim 25, wherein
the modified IND gene comprises a C to T substitution at position 247 relative to SEQ ID NO:11, and the modified IND polypeptide comprises SEQ ID NO:12;
the modified IND gene comprises a G to A substitution at position 260 relative to SEQ ID NO:13, and the modified IND polypeptide comprises SEQ ID NO:14; or
the modified IND gene comprises a G to A substitution at position 301 relative to SEQ ID NO:15, and the modified IND polypeptide comprises SEQ ID NO:16.

28. The plant of claim 25, wherein the plant comprises seedpods that require about 20 grams to about 120 grams of pulling force to shatter.

29. The plant of claim 25, wherein the plant comprises seedpods that are resistant to shatter under less than about 30 grams force.

30. Seed produced by the plant of claim 25.

31. A method for generating a modified pennycress plant having reduced seedpod shatter compared to a corresponding pennycress plant lacking the modification, the method comprising:
modifying an indehiscent (IND) gene in the pennycress genome, wherein an unmodified IND gene encodes a polypeptide having at least 95% sequence identity to SEQ ID NO:9,
generating a modified pennycress plant, wherein the modified plant exhibits reduced seedpod shatter compared to a corresponding pennycress plant lacking the modification.

32. The method of claim 31, wherein the modifying comprises site-specific gene editing, wherein the site-specific gene editing comprising introducing a guide sequence and a nuclease into a plant cell, wherein the guide sequence comprises a nucleic acid sequence specific to the IND gene, and wherein the nuclease is a CRISPR associated system (Cas) nuclease.

33. The method of claim 31, wherein the modifying comprises mutagenesis, wherein the mutagenesis comprises introducing a mutagen into a plant cell, wherein the mutagen comprises ethyl methane sulphonate (EMS).

34. The method of claim 31, wherein the modifying comprises a substitution.

35. The method of claim 31, wherein
the modified IND gene comprises a C to T substitution at position 247 relative to SEQ ID NO:11, and the modified IND polypeptide comprises SEQ ID NO:12;
the modified IND gene comprises a G to A substitution at position 260 relative to SEQ ID NO:13, and the modified IND polypeptide comprises SEQ ID NO:14; or
the modified IND gene comprises a G to A substitution at position 301 relative to SEQ ID NO:15, and the modified IND polypeptide comprises SEQ ID NO:16.

36. The method of claim 31, further comprising:
selecting a plant cell having a modification in the IND gene; and
regenerating a modified plant from the selected plant cell, wherein the plant exhibits reduced seedpod shatter.

37. The method of claim 31, wherein the modified plant comprises seedpods that require about 20 grams to about 120 grams of pulling force to shatter.

38. The method of claim 31, wherein the modified plant comprises seedpods that are resistant to shatter under less than about 30 grams force.
